(19) United States
(12) Patent Application Publication
(10) Pub. No.: US 2004/0052771 A1
(43) Pub. Date: Mar. 18, 2004

(54) Hemangioblast progenitor cells

(76) Inventor: Sai Kiang Lim, Singapore (SG)

Correspondence Address: One World Trade Center, Suite 1600, 121 S.W. Salmon Street, Portland, OR, 97204, US

(21) Appl. No.: 10/618,540

(22) Filed: Jul. 9, 2003

Related U.S. Application Data
(60) Provisional application No. 60/453,729, filed on Jul. 12, 2002.
(60) Provisional application No. 60/426,789, filed on Nov. 18, 2002.

(51) Int. Cl. A61K 45/00; C12N 5/08

(52) U.S. Cl. 424/93.7; 435/372

(57) ABSTRACT

The invention relates to isolated hemangioblast cells. Hematopoietic and endothelial cells are postulated to be derived from a common progenitor, hemangioblast. While hemangioblast has been isolated retrospectively during embryonic stem cell differentiation, it has not been isolated from embryos or from bone marrow. Prospectively stable clonal cell lines have been isolated from mammalian embryos, from embryonic stem cells and from mammalian bone marrow that can differentiate in vitro into tubular structures with both endothelial and hematopoietic markers such as CD34, CD31, Flk-1, TIE2, P-selectin, Sca-1, thy-1, CD45, and smooth muscle actin. Gene expression profiles in the undifferentiated and differentiated cells were consistent with endothelial and hematopoietic differentiation potential. Transplantation studies in isogenic or immunodeficient mice demonstrated that these cells were not tumorigenic. In an appropriate microenvironment, the cells incorporate into the vasculature and participate in hematopoiesis.

### FIELD OF THE INVENTION

The present invention relates to the derivation of hemangioblast cell lines which have the potential to differentiate into hematopoietic and endothelial cells in vitro and in vivo.

### BACKGROUND OF THE DISCLOSURE

Hematopoiesis and vasculogenesis are closely associated events that develop in tandem spatially and temporally during embryogenesis (Murray, 1932; Sabin, 1920). Primitive hematopoiesis and the establishment of the yolk sac vasculature occur simultaneously when mesodermal cells in the presumptive yolk sac proliferate and differentiate to form vascular structures with primitive erythroblasts known collectively as blood islands. Hematopoiesis during mouse development is well characterized (Keller et al., 1999). Blood islands are visible in the yolk sac at 7.5 days post coitus (dpc). By 11.5 dpc, the fetal liver displaces the yolk sac as the major site of hematopoiesis in mouse embryo and also signifies the switchover to definitive hematopoiesis. Unlike primitive hematopoiesis that is restricted to yolk sac and consists predominantly of nucleated primitive erythrocytes and some macrophages, definitive hematopoiesis encompasses all other hematopoietic activity and produces small enucleated erythrocytes.

For many years, it was accepted that the fetal liver was the site of definitive hematopoiesis in the fetus until birth, and that yolk sac hematopoietic precursor cells seeded the liver (Moore and Metcalf, 1970). However, recent studies have demonstrated that the developing dorsal aorta and surrounding area known as para-aortic splanchnopleura or aorta-gonad-mesonephros (P-Sp/AGM) precedes the fetal liver as the intra-embryonic site for the development of definitive hematopoiesis (Dzierzak, 1999). In a series of studies, it was demonstrated that as early as 8.5 dpc, the developing aorta in the mouse has multipotential hematopoietic progenitor cells and by 10.5 dpc, it has stem cells that are capable of repopulating adult recipients (Godin et al., 1995; Muller et al., 1994). These studies are significant in demonstrating that the close association between primitive hematopoiesis and endothelial development in the yolk sac also extends to definitive hematopoiesis and endothelial development of the embryonic aorta in the embryo proper. They capped a long series of observations that the embryonic development of hematopoietic and endothelial lineages are closely linked.

This close spatial and temporal association of hematopoietic and endothelial lineages during embryogenesis led to the postulation of a common progenitor for both lineages, the hemangioblast, nearly a century ago (Murray, 1932). In recent years, molecular and genetic studies demonstrating considerable concordance of molecular markers that defined both endothelial and hematopoietic cells (Keller, 2001) have also strongly supported the existence of the hemangioblast (Keller, 2001). Consistent with their common putative origin, endothelial and hematopoietic cells hematopoietic and vascular tissues express many common antigens such as flk-1, flt-1, TIE2, scl/tal-1, GATA-2 and PECAM-1, many of which are transcription factors and growth factor receptors. Targeted inactivation of some key hematopoietic or endothelial regulatory molecules such as flk-1 and its ligand, VEGF (Carmeliet et al., 1996; Ferrara et al., 1996; Shalaby et al., 1995), TIE2 (Suri et al., 1998; Takakura et al., 1998), scl/tal-1 (Robb et al., 1996) in knockout mice resulting in the disruption of both hematopoiesis and vasculogenesis further supported the hemangioblast hypothesis. These knockout mice demonstrated that differentiation of endothelial and hematopoietic cells during embryogenesis is regulated by similar genes (Keller, 2001). Disruption of either endothelial or hematopoietic development invariably involves disruption of both processes.

Keller and co-workers have demonstrated the derivation of both hematopoietic and endothelial lineages from single differentiated ES cells (Choi et al., 1998). However, the isolation of hemangioblast from embryos and its prospective isolation have hitherto remained elusive. Furthermore, a bipotential precursor cell has never been prospectively isolated. More importantly, it has never been isolated from embryos leading to the suggestion that this cell type may not exist (Keller, 2001).

U.S. Pat. No. 5,599,703 describes a method of amplifying in vitro stem cells. In this method, hematopoietic CD34.sup.+ stem and progenitor cells isolated from human bone marrow are contacted with endothelial cells, and cultured in the presence of at least one cytokine. This method produces increased yields of hematopoietic CD34.sup.+ stem and progenitor cells which can be used in human therapeutics.

U.S. Pat. No. 5,980,887 describes the use of endothelial cell (EC) progenitors in a method for regulating angiogenesis, i.e., enhancing or inhibiting blood vessel formation. For example, the EC progenitors can be used to enhance angiogenesis or to deliver an angiogenesis modulator, e.g. anti- or pro-angiogenic agents, respectively, to sites of pathologic or utilitarian angiogenesis. The patent remarks that ECs and haematopoietic stem cells (HSCs) may share a common hypothetical precursor, the hemangioblast.

U.S. published patent application SN 20020068045 relates to the production of human embryonic stem (ES) cells capable of yielding somatic differentiated cells in vitro, and committed progenitor cells such as neural progenitor cells capable of giving rise to mature somatic cells including neural cells and/or glial cells.

### SUMMARY OF THE DISCLOSURE

The present invention relates to isolated hemangioblast cell lines which have the potential to differentiate into hematopoietic and endothelial cells in vitro and in vivo. Methods are described for deriving these cell lines from mammalian embryos and from mammalian embryonic stem cells. One such cell line, RoSH2, has been deposited. As well, methods are described for deriving these cell lines from mammalian bone marrow. Three such cell lines have been established, namely Ro(BM)SH, PoSH and HuSH. Methods are also described for cultivating and propagating hemangioblast cell lines isolated according to the methods herein, and producing differentiated hematopoietic and endothelial cells therefrom.

In one aspect of the present invention, there is provided a preparation of undifferentiated mammalian hemangioblast cells capable of proliferation and differentiation in vitro and in vivo into hematopoietic and endothelial progenitor cells.

In a further aspect, there is provided a purified preparation of mammalian hemangioblast cells which (i) is capable of proliferation in an in vitro culture for more than 40 generations, (ii) does not induce tumor formation in an immunodeficent Rag1 deficient mouse, (iii) maintains the potential to differentiate to hematopoietic and endothelial cells throughout the duration of said culture, and (iv) are inhibited from differentiation when cultured on a gelatinized, feeder-free layer.

Preferably, the undifferentiated hemangioblast cells are capable of maintaining an undifferentiated state when cultured on a gelatinized feeder-free layer.

In another aspect of the present invention, there is provided an undifferentiated mammalian hemangioblast cell wherein the cell is not immunoreactive with antibodies specific for markers of pluripotent cells including CD34, PECAM-1 (or CD31), Flk-1, Tie-2, Sca-1, Thy-1 and P-selectin and wherein said cell is capable of differentiating under differentiating conditions to hematopoietic and endothelial progenitor cells.

In another aspect, there is provided an undifferentiated hemangioblast cell line capable of differentiation into hematopoietic and endothelial progenitor cells.

In one embodiment, the cell line is RoSH2 deposited at the American Type Culture Collection under #PTA-4300.

In another aspect there is provided a differentiated committed progenitor cell line that may be cultivated for prolonged periods and give rise to large quantities of progenitor cells.

In another aspect there is provided a method of preparing a mammalian hemangioblast cell line, comprising the steps of: (i) culturing a delayed mammalian blastocyst or co-culturing an early post-implantation embryo with its extra-embryonic tissues, on a fibroblast feeder layer (ii) selecting colonies of adherent fibroblastic cells with loosely attached rapidly dividing round cells having ring-like cells at their edges, and (iii) testing cells in the selected colonies for ability to differentiate into both endothelial and hematopoietic cells.

In a further aspect, there is provided a method of preparing a mammalian hemangioblast cell line, comprising the steps of: (i) culturing an embryonic stem cell-derived embryoid body, on a fibroblast feeder layer, (ii) selecting colonies of adherent fibroblastic cells with loosely attached rapidly dividing round cells having ring-like cells at their edges, and (iii) testing cells in the selected colonies for ability to differentiate into both endothelial and hematopoietic cells.

In another aspect there is provided a method of preparing a mammalian hemangioblast cell line, comprising the steps of: (i) harvesting bone marrow tissue which retains integrity in tissue clumps, (ii) culturing the bone marrow tissue on a fibroblast feeder layer, (iii) selecting colonies of adherent fibroblastic cells with loosely attached rapidly dividing round cells having ring-like cells at their edges, and (iv) testing cells in the selected colonies for ability to differentiate into both endothelial and hematopoietic cells.

This invention provides a method to generate mammalian stem cell lines with hematopoietic and endothelial potential from mammalian embryos, ES cell lines and mammalian bone marrow. The cell lines that are generated may be used for the study of the cellular and molecular biology of hematopoiesis and vasculogenesis, for the discovery of genes, growth factors, and differentiation factors that play a role in hematopoiesis and vasculogenesis, for drug discovery and for the development of screening assays for teratogenic, toxic and protective effects.

Accordingly, in another aspect, the invention provides a method for inducing formation of new blood vessels in an ischemic tissue in a patient in need thereof, comprising administering to said patient an effective amount of the purified preparation of mammalian hemangioblast cells described above to induce new blood vessel formation in said ischemic tissue.

In a further aspect, the present invention provides a method of enhancing blood vessel formation in a patient in need thereof, comprising: (i) selecting the patient in need thereof; (ii) isolating human hemangioblast cells as described above; and (iii) administering the hemangioblast cells to the patient.

In yet another aspect, the present invention provides a method for treating an injured blood vessel in a patient in need thereof, comprising: (i) selecting the patient in need thereof; (ii) isolating human hemangioblast cells as described above; and (iii) administering the hemangioblast cells to the patient.

Applicant has described herein the isolation of bipotential precursor cells from mammalian embryos, from mammalian embryonic stem cells and from mammalian bone marrow and the development of stable lines of these precursor cells. Applicant has demonstrated that these cells can differentiate into endothelial and hematopoietic lineages both in vivo and in vitro. By this criterion, applicant has, for the first time, derived hemangioblast cell lines. No equivalent primary or established cell line exists.

Uses of these cells are manifold and include the following:

1. Screening and evaluating angiogenic and anti-angiogenic factors;

2. Assessing and developing angiogenic and anti-angiogenic therapeutic protocols e.g. gene therapy, bypass surgery, tissue grafting;

3. Developing protocols for growing blood vessels in vitro for therapy; growing blood cells and platelets in vitro for transfusion therapy or making blood products e.g. haemoglobin, growth factors;

4. Tissue engineering or in vitro organogenesis;

5. Cell-based therapeutic treatment to enhance blood vessel formation or repair blood vessels for ischemic diseases such as cerebrovascular ischemia, renal ischemia, pulmonary ischemia, limb ischemia, ischemic cardiomyopathy and myocardial ischemia, injured blood vessel after balloon angioplasty or deployment of an endovascular stent; and

6. Cell-based therapeutic treatment to augment or replace or treat hematopoietic cells in hematopoietic diseases such as thalassemias, sickle cell anemia, platelet deficiency, leukemias and ADA.

### BRIEF DESCRIPTION OF THE DRAWINGS

FIG. 1. Isolation of RoSH2 cell line.

(A) Sub-confluent culture of RoSH2 cells on gelatinized tissue culture plate. Cell morphology includes adherent fibroblast-like cells and ring-like structures.

(B) At confluency, the cells formed cord-like structures.

(C) RoSH2 cells stained positive for E.coli β-gal activity. The cells were incubated with Imagene Green™, a green fluorescent substrate for β-gal and counterstained with DAPI, a blue fluorescent nuclear stain.

(D) Immunohistochemistry staining of confluent RoSH2 cell culture for vWF. Arrows indicate brown positive staining. The cells were counterstained with eosin.

(E) Chromosome number. RoSH2 cells at passage 10 and passage 150 were treated with colcemid to prepare metaphase chromosomal spread. The number of chromosomes in 40 metaphase nuclei was counted.

(F) Y-chromosome FISH to demonstrate a single green fluorescent Y chromosome per nucleus (arrowhead indicates Y chromosome).

(G) PCR amplification of RoSH2 genomic for the presence of SRY gene.

FIG. 2. In vitro differentiation of RoSH2 cells. RoSH2 cells were induced to differentiate in vitro by plating the cells on matrigel.

(A) RoSH2 cells on matrigel coated plate for i) 2 days and ii) 7 days after plating.

(B) RoSH2-derived tubular mesh. One week after plating on matrigel coated plate, a mesh of tubular structures formed and began detaching from the bottom of the plate while still tightly anchored to the plate at the perimeter. The mesh was removed by rimming the plate with a 21G needle and fixed in formalin. i) The mesh was mounted on slides and stained with H&E (10× magnification). ii), iii) Paraffin embedded mesh was sectioned at 4 μm before staining with H&E and viewed under ii) 40× and iii) 60× magnification. The letter L indicates the lumen of the structure, M indicates the matrix and S indicates sprouting from the tubular structure.

(C) Confocal microscopy of tubular structure. The culture of tubular mesh was labelled with a β-gal substrate, green fluorescent Imagene Green™ and propidium iodide as described herein, and analyzed by confocal microscopy. The diameter of the S patent lumen was estimated to be 100 μM.

(D) Electron microscopy of the tubular structures. i) Endothelial cell resting on an acellular matrix with polarized plasma membrane, ii) filamentous structures on the luminal surface of endothelial cell with underlying microvesicles (arrowheads), iii) tight apposing neighboring cells, iv) electron-dense nascent Weibel-Palade bodies (arrowhead).

FIG. 3. Antigen profiles.

(A) FACS analysis of undifferentiated RoSH2 cells. (B) Confocal microscopy of RoSH2-derived tubular structures incubated with biotinylated antibodies to Flk-1, Sca-1, CD31 and CD45, stained with strepavidin-FITC and counterstained with propidium iodide.

(C) Immunohistochemistry on paraffin-embedded sections of RoSH2-derived tubular structures using HRP-based conjugated antibodies to Tie-2, Thy-1, CD34, P-Selectin and SMA. Brown precipitates indicate positive staining. The nuclei were stained with Mayer's hematoxylin.

FIG. 4. Gene Expression Profile.

(A) Sequences of RT-PCR and nested PCR primers for the different genes analyzed and expected sizes of RT-PCR products.

(B) RT-PCR Total RNA was prepared from undifferentiated RoSH2 cells, RoSH2 cells that were plated on matrigel for 24 hours and mesh of RoSH2-derived tubular structures. The total RNA was reverse transcribed into cDNA and the cDNA amplified using gene-specific probes. Triose phosphate isomerase, a housekeeping gene was used as a control.

(C) Nested RT-PCR.

FIG. 5. Endothelial cell differentiation in vivo.

(A) Anti-fas induced liver injury. RoSH2 cells were injected intrasplenically into mice treated with a-fas antibody to induce apoptosis in hepatocytes resulting in fuliminat hepatitis. After 5 days, the mouse was perfusion fixed, the liver was stained with X-gal or cryosectioned at 15 μM before staining with X-gal. The cells were counterstained with eosin.

(B) Formation of ES/RosH2 hybrid teratoma. Mouse ES cells and RoSH2 cells were co-injected subcutaneously into Rag1-/-mice to generate teratomas. After three weeks, the mice were sacrificed, perfusion-fixed and the tumors cryosectioned. RoSH2-derived cells were identified by the presence of β-gal using a HRP-based anti-β-gal antibody. Dark brown staining (arrowheads) indicates positive staining.

FIG. 6. Hematopoietic cell differentiation in vivo.

(A) Derivation of T-cells from ROSH cells. RoSH2 cells were injected intraperitoneally into Rag1-/-mice. After 6 months, the spleens were harvested and stained for the presence of CD3+ cells using a HRP-based anti-CD3 antibody. Brown membrane staining (arrow) indicates positive CD3 staining. The cells were counterstained with H&E.

(B) Derivation of erythrocytes and megakaryocytes. RoSH2 cells were injected intraperitoneally into 5FU-treated mice as described herein. After ten weeks, spleens were removed from surviving mice and assayed for colony forming units. Colonies were picked and the cells were stained with Imagene Green™ for the presence of β-gal and counterstained with propidium iodide. i) Host erythrocytes, ii) RoSH2-derived β-gal positive erythrocytes. Arrows indicate enucleating erythrocytes and arrowheads indicate enucleated erythrocyte. iii) Host megakaryocytes. iv) RoSH2-derived β-gal positive megakaryocytes. Arrowheads indicate multinucleated megakaryocytes.

FIG. 7. In vitro differentiation and gene expression profiles of E-RoSH1 cells (which are ES cell-derived RoSH-like cells).

(A) E-RoSH1 cells were induced to differentiate in vitro by plating the cells on matrigel. A mesh of patent tubular structures formed after a week. The tubular structures were incubated with red fluorescent diI labelled acetylated LDL overnight, fixed in formalin and counterstained with SYTOX Green, a green fluorescent dye for nuclei.

(B) Gene Expression Profile of RoSH2 and E-RoSH1 by RT-PCR. Total RNA was prepared from undifferentiated embryo-derived RoSH2 cells and ES cell-derived E-RoSH1 cells. The total RNA was reverse transcribed into cDNA and the cDNA amplified using genespecific probes. Triose phosphate isomerase, a housekeeping gene, was used as a control.

FIG. 8. Isolation of Ro(BM)SH cells. Sub-confluent culture of Ro(BM)SH cells on gelatinized tissue culture plate. Cell morphology includes adherent fibroblast-like cells and ring-like structures.

FIG. 9. FACS analysis of Ro(BM)SH cells for CD34, PECAM-1 (or CD31), Flk-1, TIE2, Sca-1, Thy-1, CD45 and P-selectin markers of pluripotent cells. The proportion of cells that were positive for these markers corresponded with the approximate proportion of ring-like cells in the cell population, suggesting that these markers were detectable only on differentiated cells.

FIG. 10. Gene Expression Profile of Ro(BM)SH by RT-PCR. Total RNA was prepared from undifferentiated bone marrow-derived Ro(BM)SH cells. The total RNA was reverse transcribed into cDNA and the cDNA amplified using gene-specific probes. Triose phosphate isomerase, a housekeeping gene, was used as a control.

### DETAILED DESCRIPTION

Applicant has described the isolation and establishment of hemangioblast progenitor cell lines from mammalian embryos, from mammalian embryonic stem cells and from mammalian bone marrow, which have the potential to differentiate into both hematopoietic and endothelial cells. The establishment of monoclonality in these cell lines is preferred to obtain cell lines that have this bi-potentiality. The procedures are described which the applicant has taken at several stages of isolation to ensure monoclonality.

During isolation from mammalian embryos, single colonies were selected from primary blastocyst or embryo cultures whenever possible. During isolation from mammalian bone marrow, single colonies were selected from individual bone marrow clumps whenever possible. In other situations, single cells were plated in 96-well plates by limiting dilution. Once a line was established, care was taken to clone the line by plating single cells in methlycellulose-based media. The establishment of a hemangioblast cell line is defined by its ability to propagate hemangioblast cells in culture continuously for more than 40 generations without loss of proliferation activity and phenotype. Like most murine ES cell lines, applicant's hemangioblast cell lines were observed to have a XY karyotype and a normal euploid chromosome number (eg, 40 in mouse). Long-term propagation of these cells in culture resulted in a loss of chromosomes.

Pluripotent ES cell lines routinely can be derived from both earlier and delayed blastocysts (eg from 3.5 dpc through delayed 5.5 dpc mouse blastocysts). In contrast, applicant's embryo-derived hemangioblast cell lines are derived from delayed blastocysts (eg 5.5 dpc mouse blastocysts or other mammalian equivalent delayed blastocyst). The preferential derivation of hemangioblast cell lines from delayed blastocysts contrasts with the routine derivation of pluripotent ES lines. The hemangioblast cells appear to be more lineage-restricted than ES cells and may not have been generated in earlier (eg 3.5 dpc mouse) blastocysts. For hemangioblast cell lines derived from embryonic stem cells, the timing appears to be cell type dependent.

Consistent with these observations, applicant has injected subcutaneously three of its independently derived hemangioblast cell lines into immunodeficient Rag-/-mice or isogenic C57BL/6J newborn or adult mice, and has not observed the formation of teratomas during a six-month observation period. Applicant's mouse hemangioblast cells proliferate in culture with an average population doubling time of 12-15 hours that is reminiscent of mouse ES cells. Based on the derivation of the cells from early embryos or from embryonic stem cells, and their observed characteristics, applicant has concluded that these cells are hemangioblasts, or primitive, multipotential stem cells.

Potential hemangioblast cells can be isolated from mammalian embryos, embryonic stem cells or mammalian bone marrow. The isolated potential hemangioblast cells are plated and cultured. Egg cylinders derived from later stage embryos (eg 6 to 7.5 dpc mouse blastocysts) were placed next to extra embryonic tissues for culture. Potential hemangioblast cells are found in colonies having at least some cells which exhibit a specific morphology, including adhesive fibroblast-like and ring-like structures.

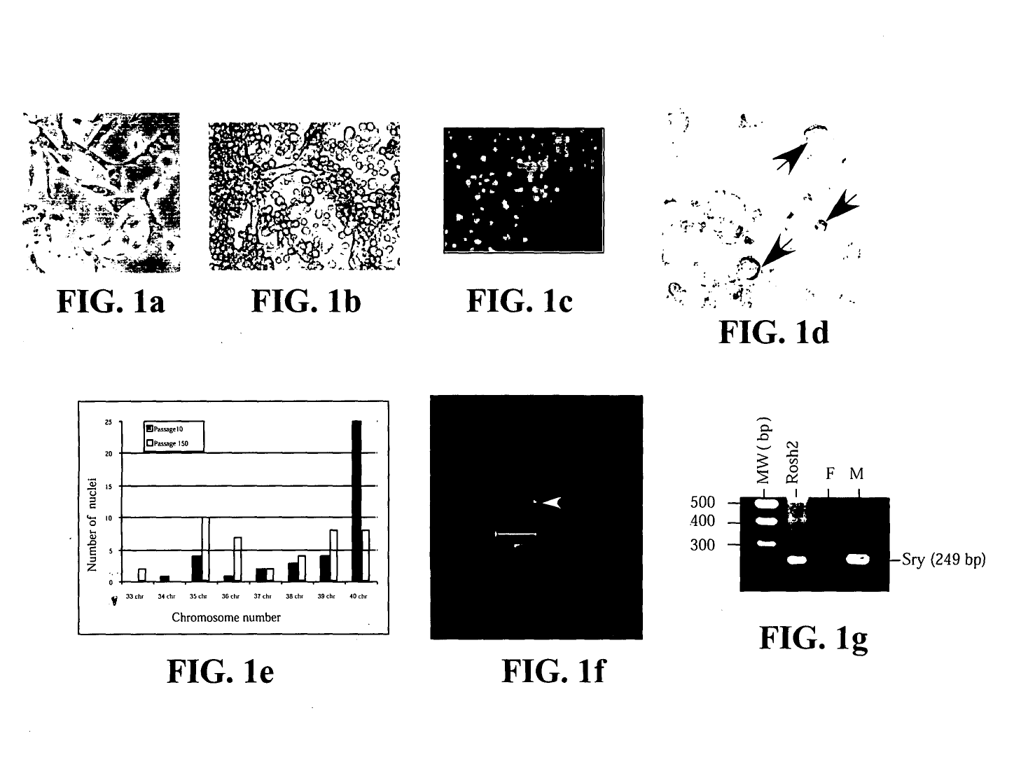

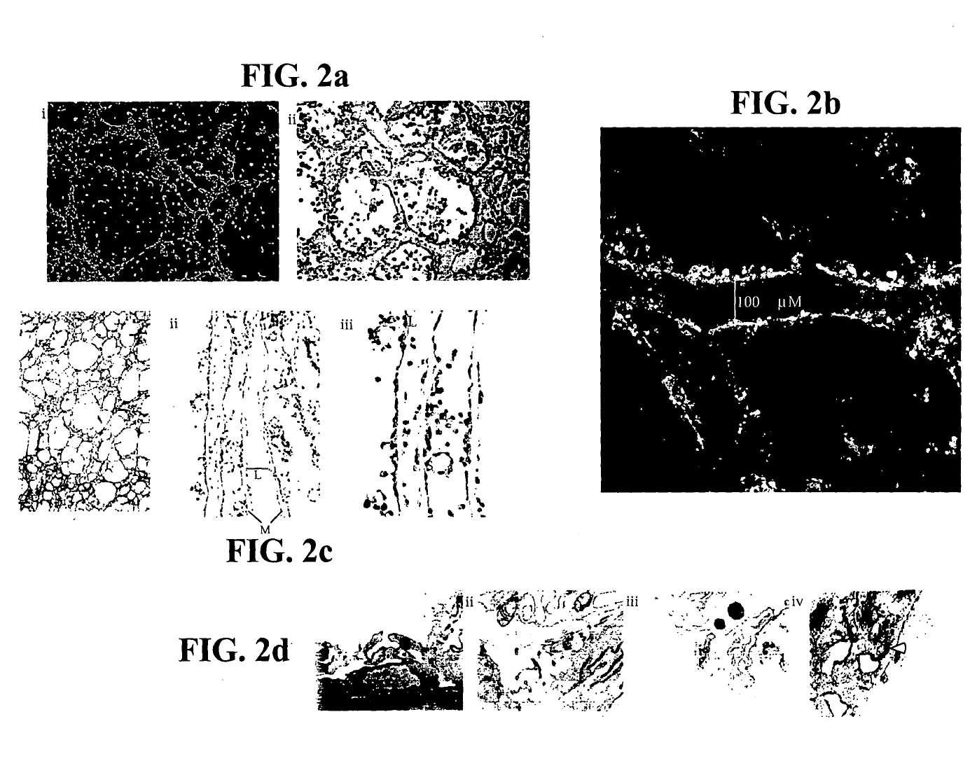

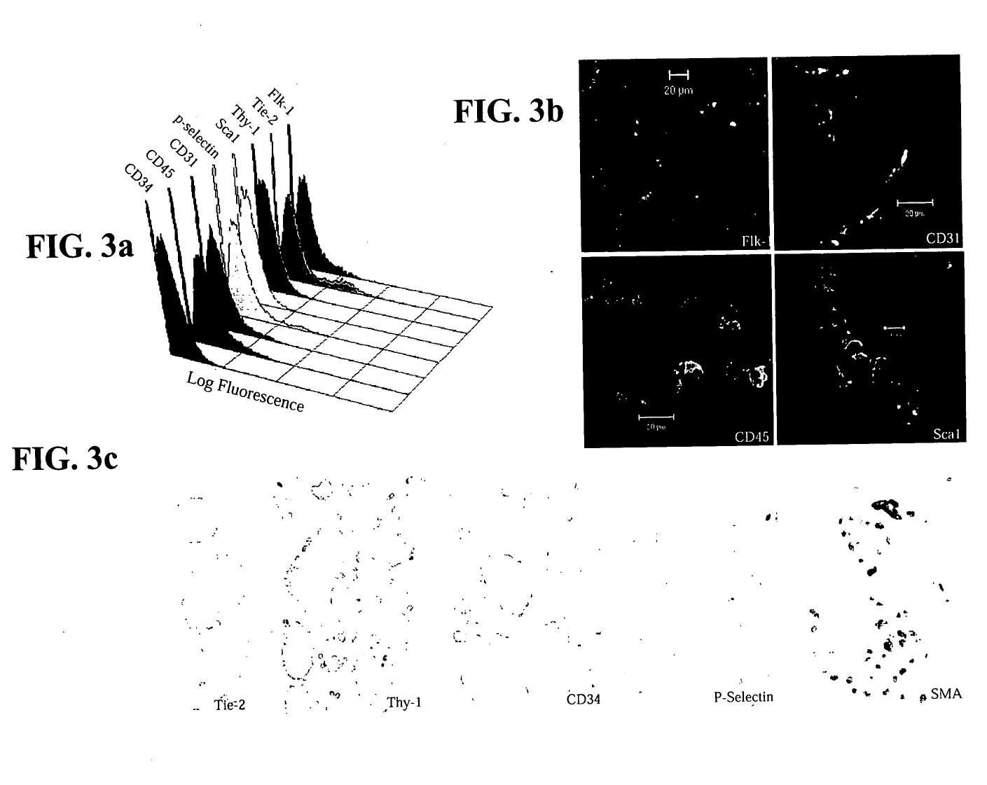

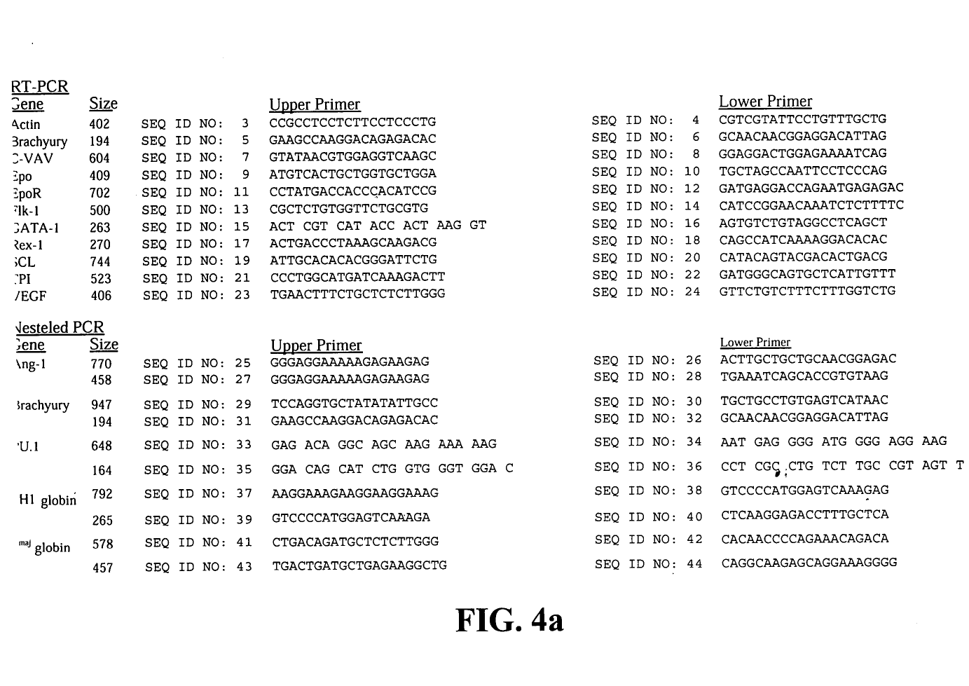

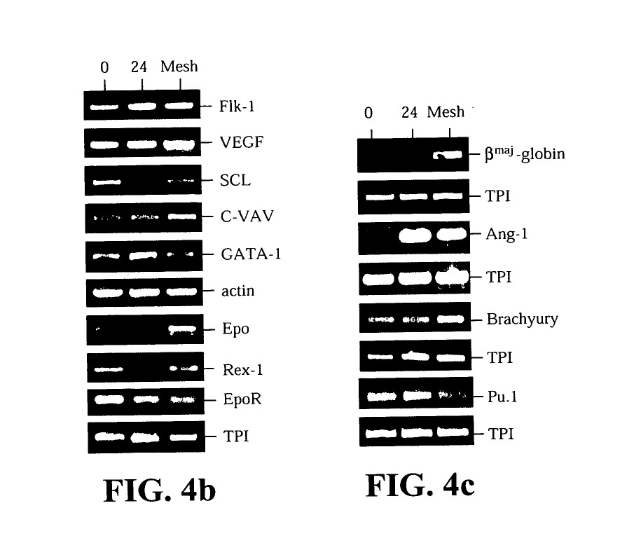

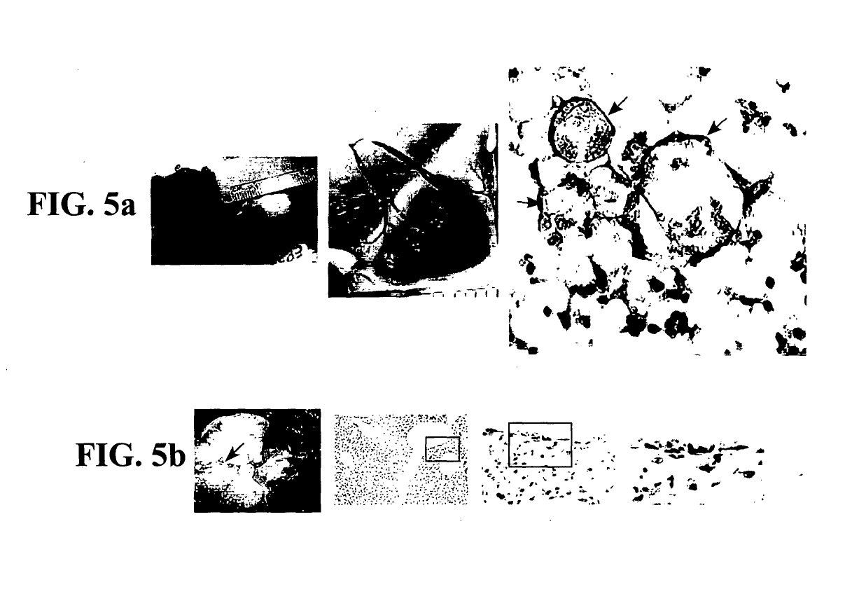

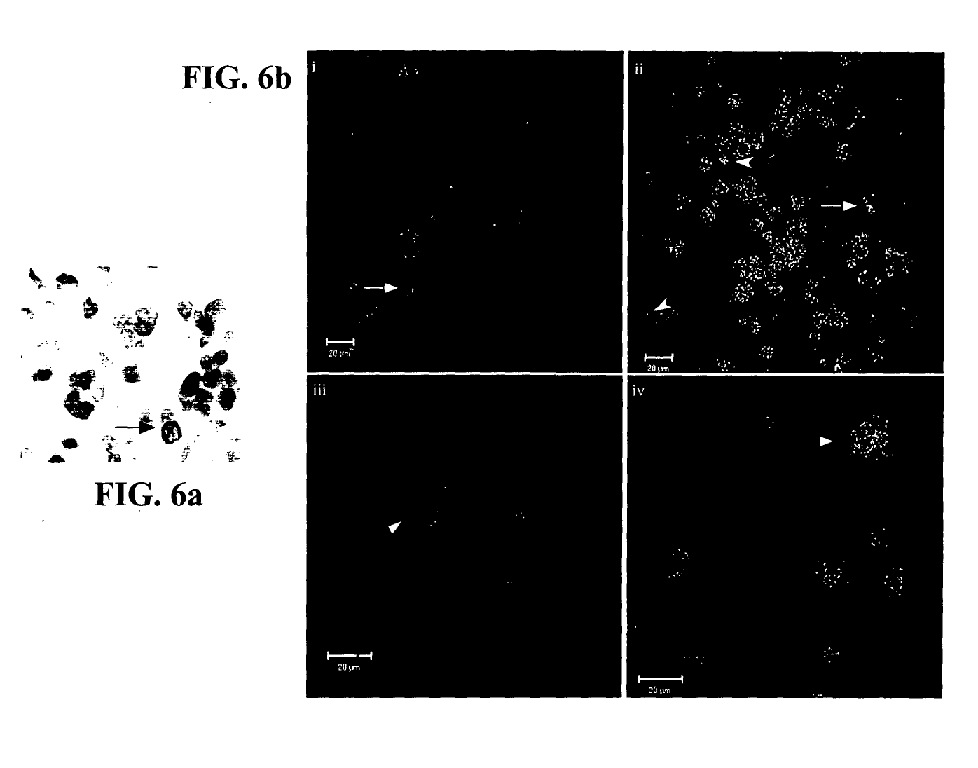

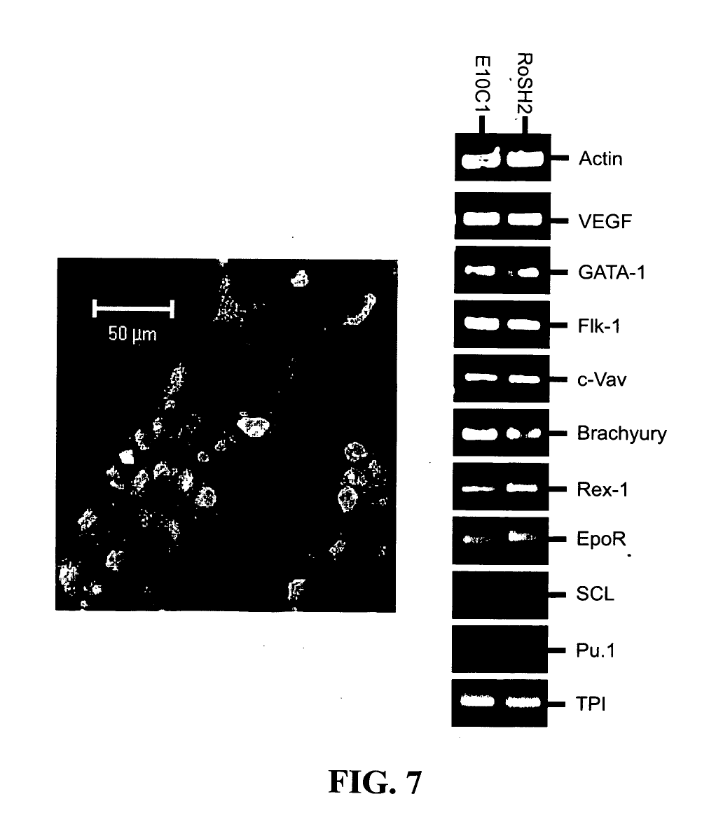

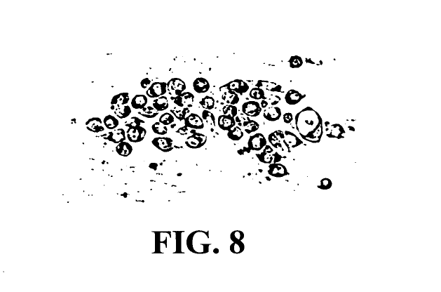

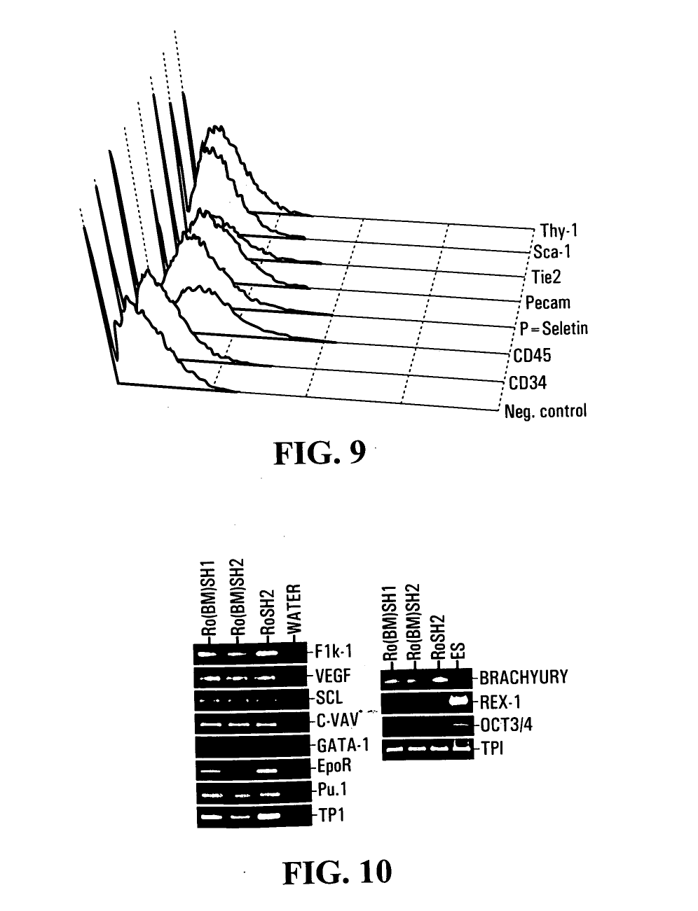

Colonies are selected which display, at the edge of the colony, some spontaneously differentiated cells having a distinct ring-like structure (FIG. 1A). Undifferentiated cells in the center of colonies so identified were then selected to establish hemangioblast cell lines. Alternatively, the entire colonies so identified can be selected to establish hemangioblast cell lines since the spontaneously differentiated ring-like cells will die off eventually.

To assess the relatedness of embryo-derived hemangioblast cells or bone marrow-derived hemangioblast cells to other primitive cells such as ES cells or the epiblast and to mesodermal lineage, the presence of Rex-1 and Brachyury gene expression was assayed for. Rex-1 is a zinc-finger transcription factor that is regulated by Oct3/4 and its expression is restricted to ES cells, ICM cells and spermatocytes (Rogers et al., 1991). Brachyury is a T box gene that is expressed in the presumptive mesoderm of the late blastula and functions in early mesodermal specification (Herrmann and Kispert, 1994; Smith, 1997; Technau, 2001). The expression of both Rex-1 and Brachyury in embryo-derived hemangioblast cells suggests that these cells are at the threshold of mesodermal commitment from the epiblast stage, i.e. embryo-derived hemangioblast cells are early embryonic cells and embryo-derived and bone marrow-derived hemangioblast cells are likely to be multipotent. Further, upon FACS analysis, applicant's hemangioblast cells do not display markers such as CD34, PECAM-1 (or CD31), Flk-1, Tie-2, Sca-1, Thy-1 or P-selectin, which are common to pluripotent cells. This observation is consistent with a recent report that multipotent adult progenitor cells from bone marrow are CD34, CD44, CD45, c-Kit and MHC class I and II negative, and have low levels of Flk-1, Sca-1 and Thy-1 (Jiang, Y., et al. (2002)).

A significant indicator of cell colonies containing hemangioblast cells is the spontaneous formation of ring-like cells that are strongly vWF immunoreactive. At high confluency on gelatinized plates, the formation of cordlike-structures was observed. When plated on matrigel, the cells assembled to form a mesh of patent tube-like structures. Using several different criteria such as electron microscopy, immunohistochemistry and gene expression profiles, the mesh of patent tube-like structures was demonstrated to be composed of endothelial cells in a highly typical 3D-structural organization of vascular tissue. These cells displayed typical endothelial and hematopoietic markers such as CD34, PECAM-1 (or CD31), Flk-1, Tie-2, Sca-1, Thy-1 and P-selectin. More importantly, hematopoietic cells as defined by the presence of surface CD45 and putative pericytes as indicated by the presence of cytoplasmic SMA were also present.

Consistent with immunohistochemical analysis, it was also determined that genes essential for differentiation and maintenance of endothelial or hematopoietic lineages were expressed in embryo-derived hemangioblast cells (e.g. Flk-1, VEGF, angiopoietin-1, c-vav, GATA-1, erythropoietin, erythropoietin receptor, PU.1, β^{maj}-globin, and SMA) and in bone marrow-derived hemangioblast cells (e.g. Flk-1, VEGF and erythropoietin receptor). Flk-1 is the receptor for VEGF, a critical endothelial growth factor. Both Flk-1 and VEGF are essential for vasculogenesis and angiogenesis during embryo development (Carmeliet et al., 1996; Ferrara et al., 1996; Shalaby et al., 1995). As evidenced by gene knockout experiments in mice, inactivation of either both alleles of Flk-1 or one allele of VEGF cause severe disruption of vasculogenesis and hematopoiesis (Carmeliet et al., 1996; Ferrara et al., 1996; Shalaby et al., 1995). TIE2 is a receptor tyrosine kinase for angiopoietin-1 and is expressed almost exclusively in endothelial cells and early hematopoietic cells (Davis et al., 1996). Unlike Flk-1/VEGF receptor-ligand complex, TIE2/Angiopoietin-1 receptor-ligand complex does not directly promote the growth of cultured endothelial cells but is required for the later stages of vascular remodelling and definitive hematopoiesis (Suri et al., 1998; Takakura et al., 1998). The expression of VEGF in embryo-derived and bone marrow-derived hemangioblast cells explains the spontaneous differentiation of those cells into endothelial cells when cultured on matrigel without addition of exogenous VEGF. In this regard, embryo-derived and bone marrow-derived hemangioblast differs from the ES-derived hemangioblast that requires VEGF for endothelial differentiation. (Choi, 1998; Kennedy et al., 1997). Consistent with the role of angiopoietin-1 in the later stages of vascular remodelling and definitive hematopoiesis, its expression was limited to later stages of embryo-derived hemangioblast differentiation into tubular structures. It is likely that the extensive branching and sprouting of tubular structures observed during in vitro differentiation of embryo-derived hemangioblast cells could be attributed in part to the expression of angiopoietin-1.

Scl/tal-1, a helix-loop-helix transcription factor that is expressed in vascular endothelium and hematopoietic cells, was also expressed in embryo-derived hemangioblast cells. Although it has been shown to be dispensable for endothelial differentiation, it is required for angiogenic modeling in the embryo and is absolutely required for commitment of a putative hemangioblast to the hematopoietic lineage (Robb et al., 1996). A downregulation followed by an upregulation of scl/tal-1 expression was observed during differentiation of RoSH2, an embryo-derived hemangioblast cell line (FIG. 4B). It is not clear if this downregulation coincided with predominant commitment of ROSH cells to endothelial lineage during the initial part of the differentiation program and the subsequent upregulation is associated with a subsequent increase in hematopoietic commitment. Nevertheless, the expression of scl/tal-1 in ROSH cells during differentiation is highly consistent with the characterization of ROSH as a putative hemangioblast capable of differentiating into hematopoietic and endothelial lineages.

Although the expression of scl/tal-1 is strongly supportive of hematopoiesis, an important corollary that will verify hematopoietic differentiation of RoSH2 is the expression of downstream scl/tal-1-regulated hematopoietic genes such as PU.1 and GATA-1. PU.1 is an ets transcription factor and is an important regulator of B lymphoid- and myeloid-specific genes (Hromas et al., 1993; Nerlov and Graf, 1998; Scott et al., 1994). Targeted inactivation of PU.1 causes embryonic lethality with a severe defect in the generation of progenitors for B and T lymphocytes, monocytes, and granulocytes (McKercher et al., 1996; Scott et al., 1994). The expression of PU.1 suggests that RoSH2 cells are capable of differentiating into lymphocytes and consistent with this hypothesis, vav was also expressed. Vav is a guanine nucleotide exchange factor essential for T and B lymphocyte signalling as evidenced by defective T and B lymphocyte signalling in gene knockout experiments (Fischer et al., 1995; Tarakhovsky et al., 1995). It is expressed in both fetal and adult hematopoietic cells of all lineages (Bustelo et al., 1993; Katzav et al., 1989) and in a limited number of non-hematopoietic cells or tissues such as ES cells, the developing tooth, testicular germ cells and trophoblast (Bustelo et al., 1993; Keller et al., 1993). The expression of both PU.1 and vav in RoSH2 suggests that ROSH cells are capable of differentiating into lymphocytes with the inherent implication that RoSH2 cells have a propensity towards definitive and not primitive hematopoietic differentiation.

GATA-1, a scl/tal-1-dependent zinc-finger transcription factor, is highly critical in primitive and definitive erythropoiesis (Pevny et al., 1991; Simon et al., 1992). Its expression suggests that RoSH cells have the capability to undergo erythropoiesis. Applicant has also observed that RoSH2 cells have a propensity towards definitive and not primitive hematopoietic differentiation, which suggests that ROSH cells are more likely to undergo definitive erythropoiesis. This was confirmed by the expression of adult β^{maj}-globin mRNA and not the embryonic βh1-globin. The expression of erythropoietin and erythropoietin receptor was also consistent with definitive erythropoiesis. Erythropoietin receptor is expressed in both primitive and definitive erythropoietic tissues but erythropoietin whose expression in the adult kidney is well documented, is also expressed in definitive hematopoeitic stem cells but not primitive erythropoietic tissues. Targeted inactivation of erythropoietin receptor results in defective definitive erythropoiesis with no obvious defects in primitive erythropoiesis (Lin et al., 1996; Wu et al., 1995). Therefore, the expression of both erythropoietin and erythropoietin receptor fulfils a minimal requirement for definitive erythropoiesis to occur and is consistent with the process of definitive hematopoiesis.

The in vitro data was further verified by in vivo transplantation studies. Using several experimental models, it was shown that embryo-derived hemangioblast cells participated in angiogenesis during anti-fas mediated liver injury and healing, and vascularization of ES cell-derived teratomas. It is noteworthy that RoSH2 cells, an embryo-derived hemangioblast cell line, will proliferate and differentiate into endothelial cells only under an angiogenic microenvironment. To verify the hematopoietic potential of embryo-derived hemangioblast cells, ROSH cells were demonstrated to differentiate into CD3+ mature T-cells in Rag1-/-mice that are deficient in CD3+ mature T-cells, and erythrocyte and megakaryocyte progenitor cells in 5FU-treated mice. Together with the data from in vitro studies, applicant has provided compelling evidence of the reproducible isolation of embryo-derived hemangioblast cells capable of differentiating into both endothelial and hematopoietic cells.

Applicant also has derived hemangioblast cell lines from embryonic stem (ES) cells. These ES cell-derived cell lines are morphologically similar to and have similar differentiation potential as the above-described embryo-derived hemangioblast cell lines. Using ES cells rather than embryos to derive hemangioblast is particularly useful for deriving human hemangioblast cell lines, where, for ethical reasons, it may be preferable to derive human hemangioblast cell lines from available human embryonic stem cell lines rather than from human embryos. ES cell-derived embryoid bodies (EBs) are known to recapitulate early embryos (Doetschman et al., 1985) and have been shown to produce hemangioblast (Choi K et al., 1998).

Consistent with applicant's observations that delayed 5.5 dpc mouse blastocysts to 7.5 dpc embryos were most efficient for derivation of hemangioblast lines, Choi et al. have previously determined that day 6 (DG) EBs are most efficient in generating hemangioblasts (Choi K et al., 1998). D6 EBs are analogous to early post-implantation embryos. There are several published methods for preparing embryoid bodies (Wiles et al., 1993). The method used by the applicant is described in Example 2 herein. ES cells were cultured in semi-solid methycellulose media. Colonies of cells or EBs were clearly visible to the naked eye. The EBs were dissociated into cell suspensions. The cells were then plated and allowed to proliferate and differentiate into a complex mixture of cell types. Colonies of rapidly dividing cells resembling embryo-derived hemangioblast cells were selected based on the morphological characteristics described above for embryo-derived hemangioblast cells. A number of hemangioblast cell lines were established from these colonies in the same fashion as for the embryo-derived hemangiobalst cell lines.

Alternatively, another method of isolating hemangioblast cell lines from EBs is to select individual EBs and place one EB per well in a gelatinized 96-well feeder plate. Each EB then adheres to the culture dish. In most instances, the EBs will proliferate into a complex mixture of cells. After several days of proliferation, putative hemangioblast colonies are usually present in >50% of the wells. By serially expanding these cells on feeder cells, a majority of cell cultures reach senescence and die. About 50% of the surviving cell cultures are hemangioblast cultures that have round, rapidly dividing cells and characteristic fibroblastic cells at the periphery of which are cells having ring-like structures.

The age of EBs that are most efficient for the derivation of hemangioblast lines varies with the parental ES lines. For example, D3 to D5 EBs derived from E14 ES cell line and D6 EBs derived from CS1 ES cell line both are very efficient for obtaining ES cell-derived hemangioblast cell lines. Although applicant has successfully derived hemangioblast lines from spontaneously differentiating ES cells grown in the absence of LIF or other developmental stages of EB or EBs grown in suspension cultures, the efficiency is much lower and less reproducible.

ES cell-derived hemangioblast lines are morphologically similar to embryo-derived hemangioblast cell lines with similar culture conditions and a population doubling time of 15 hours. Applicant has derived a number of ES cell-derived hemangioblast lines from CS1 ES and E14 ES murine cell lines. They have a normal chromosomal number of 40 and have been maintained in continuous culture for more than 40 generations. They can be induced to differentiate to form endothelial vessels by plating on matrigel and like typical endothelial cells, they endocytose acetylated LDL (FIG. 7A). Like embryo-derived hemangioblast cell lines, these cells express both endothelial and hematopoietic specific genes such as smooth muscle actin, VEGF, GATA-1, Flk-1, c-vav, EpoR, SCL/tal-1 and Pu.1 (FIG. 7B). ES cell-derived hemangioblast cells also express Rex-1 and Brachyury gene suggesting that, like embryo-derived hemangioblast cells, they have retained some features of pluripotent ES cells such as the expression of Rex-1 and have also committed to mesodermal lineage. Applicant has therefore demonstrated that murine hemangioblast cell lines can be derived from EBs derived from ES cells. The same method can be used to obtain hemangioblast cell lines from other mammalian embryos and embryonic stem cells. In particular, since human ES cell lines have been shown to form EBs and generate endothelial progenitor cells (Levenberg et al., 2002), hemangioblast lines can be derived from human ES cells in the same way.

In conclusion, applicant has demonstrated that monoclonal embryo-derived ROSH cells and morphologically similar ES-derived cells are lineage restricted stem cells with the capability of differentiating into endothelial and hematopoietic cells in vitro and in vivo. Applicant has also demonstrated that monoclonal bone marrow-derived Ro(BM)SH, PoSH and HuSH cells are lineage restricted stem cells with the capability of differentiating into endothelial and hematopoietic cells. By this criterion, these cells qualify as hemangioblast. The potency of applicant's RoSH2 cell line has been demonstrated in at least two different lineages.

The ROSH, Ro(BM)SH, PoSH and HuSH cell lines established by the applicant provide a useful reference for the characterization of hemangioblast. Hemangioblast cell lines are useful in characterizing or identifying early molecular events and molecules or factors in lineage commitment, differentiation and tissue organization during vasculogenesis, angiogenesis and hematopoiesis.

Since mammals share a highly conserved developmental plan during embryogenesis particularly at the embryonic pre- and early post-implantation stages from which ROSH cell lines were established, ROSH homologous hemangioblast cell lines can be isolated from other mammalian embryos and embryonic stem cells using the procedure described above. As well, Ro(BM)SH, PoSH and HuSH homologous hemangioblast cell lines can be isolated from other mammalian bone marrow using the same procedure as described above for Ro(BM)SH, PoSH and HuSH cell lines.

Since the hemangioblast cells obtained by the methods of this invention from mammalian embryos, ES cell lines and mammalian bone marrow have dual potential to differentiate into hematopoietic and endothelial cells, the cell lines that are generated may be used for the study of the cellular and molecular biology of hematopoiesis and vasculogenesis, for the discovery of genes, growth factors, and differentiation factors that play a role in hematopoiesis and vasculogenesis, for drug discovery and for the development of screening assays for teratogenic, toxic and protective effects.

Since the hemangioblast cells of the invention will proliferate and differentiate into endothelial cells under an angiogenic microenvironment, the hemangioblast cells may also be used in a therapeutic manner to provide new blood vessels or to induce repair of damaged blood vessels at a site of injury in a patient. Accordingly, the invention provides various methods involved in providing blood vessel growth or repair to a patient in need thereof. In one aspect, the invention provides a method for inducing formation of new blood vessels in an ischemic tissue in a patient in need thereof, comprising administering to said patient an effective amount of the purified preparation of mammalian hemangioblast cells described above to induce new blood vessel formation in said ischemic tissue. In a further aspect, the present invention provides a method of enhancing blood vessel formation in a patient in need thereof, comprising: (i) selecting the patient in need thereof; (ii) isolating human hemangioblast cells as described above; and (iii) administering the hemangioblast cells to the patient. In yet another aspect, the present invention provides a method for treating an injured blood vessel in a patient in need thereof, comprising: (i) selecting the patient in need thereof; (ii) isolating human hemangioblast cells as described above; and (iii) administering the hemangioblast cells to the patient.

Hemangioblast cell lines are also useful in understanding organogenesis. During development, the early developing endothelial cells and their precursors have been shown to be crucial in organogenesis (Bahary and Zon, 2001). Recently, two studies demonstrated that the developing endothelium of the embryonic dorsal aorta is critical in inducing the development of the pancreas and liver, possibly through the secretion of factors (Lammert et al., 2001; Matsumoto et al., 2001). Therefore, the ability to induce RoSH2 cells to undergo vasculogenesis in vitro permits the characterization and isolation of the inducing factors and the assessment of the microenvironment and interaction between endothelium and mesoderm or ectodermal tissues during organogensis.

Hemangioblast cell lines may also be used in gene therapy. Generally, the preparation of mammalian hemangioblast cells of the invention may be used to deliver a therapeutic gene to a patient that has a condition that is amenable to treatment by the gene product of the therapeutic gene. The hemangioblasts are particularly useful to deliver therapeutic genes that are involved in or influence angiogenesis (e.g VEGF to induce formation of collaterals in ischemic tissue), hematopoiesis (e.g. erythropoietin to induce red cell production), blood vessel function (e.g. growth factors to induce proliferation of vascular smooth muscles to repair aneurysm) or blood cell function (e.g. clotting factors to reduce bleeding) or code for secreted proteins e.g. growth hormone. Methods for gene therapy are known in the art. See for example, U.S. Pat. No. 5,399,346 by Anderson et al. A biocompatible capsule for delivering genetic material is described in PCT Publication WO 95/05452 by Baetge et al. Methods of gene transfer into bone-marrow derived cells have also previously been reported (see U.S. Pat. No. 6,410,015 by Gordon et al.). The therapeutic gene can be any gene having clinical usefulness, such as a gene encoding a gene product or protein that is involved in disease prevention or treatment, or a gene having a cell regulatory effect that is involved in disease prevention or treatment. The gene products should substitute a defective or missing gene product, protein, or cell regulatory effect in the patient, thereby enabling prevention or treatment of a disease or condition in the patient.

Accordingly, the invention further provides a method of delivering a therapeutic gene to a patient having a condition amenable to gene therapy comprising: (i) selecting the patient in need thereof; (ii) modifying the preparation of claim 1 so that the cells of the preparation carry a therapeutic gene; and (iii) administering the modified preparation to the patient. The preparation may be modified by techniques that are generally known in the art. The modification may involve inserting a DNA or RNA segment encoding a gene product into the mammalian hemangioblast cells, where the gene enhances the therapeutic effects of the hemangioblast cells. The genes are inserted in such a manner that the modified hemangioblast cell will produce the therapeutic gene product or have the desired therapeutic effect in the patient's body. The hemangioblast cells may be prepared from a cell source originally acquired from the patient, such as bone marrow. The gene can be inserted into the hemangioblast cells using any gene transfer procedure, for example, direct injection of DNA, receptor-mediated DNA uptake, retroviral-mediated transfection, viral-mediated transfection, non-viral transfection, lipid based transfection, electroporation, calcium phosphate mediated transfection, microinjection or proteoliposomes, all of which may involve the use of gene therapy vectors. Other vectors can be used besides retroviral vectors, including those derived from DNA viruses and other RNA viruses. As should be apparent when using an RNA virus, such virus includes RNA that encodes the desired agent so that the hemangioblast cells that are transfected with such RNA virus are therefore provided with DNA encoding a therapeutic gene product.

In accordance with another aspect of the invention, a purified preparation of mammalian hemangioblast cells, in which the cells have been modified to carry a therapeutic gene, may be provided in containers or commercial packages that further comprise instructions for use of the preparation in gene therapy to prevent and/or treat a disease by delivery of the therapeutic gene. Accordingly, the invention further provides a commercial package comprising a preparation of mammalian hemangioblast cells of the invention, wherein the preparation has been modified so that the cells of the preparation carry a therapeutic gene, and instructions for treating a patient having a condition amenable to treatment with gene therapy.

The present invention will now be more fully described with reference to the following examples, which are illustrative only and should not be considered as limiting the invention described above. In particular, the invention is illustrated by deriving hemangioblast cells from mouse embryos and embryonic stem cells. The invention is also illustrated by deriving hemangioblast cells from mouse, pig and human bone marrow. However, these examples are illustrative only and hemangioblast cells can be derived in the same fashion as exemplified herein from other mammalian embryos and embryonic stem cells, particularly human embryos and embryonic stem cells, and from other mammalian bone marrow.

### EXAMPLE 1

Materials and Methods

Derivation of RoSH2 cells: All animal experimentation protocols were approved by National University of Singapore Animal Ethics Research Committee. B6.129S7-GtRosa26 mice were purchased from Jackson Laboratory (Bar Harbor, Me.). 5.5 dpc delayed blastocysts and 6 to 7.5 dpc embryos were prepared as previously described (Robertson, 1987). For 6 to 7.5 dpc embryos, the egg cylinders were dissected out and were placed next to the extra-embryonic tissues for culture. The embryos were cultured on tissue culture dish in ES cell media. For the older embryos, the extra-embryonic tissues were removed after the embryos attached and started proliferation. For 5.5 dpc delayed blastocysts, when the growths reached a size visible to the naked eye, they were disaggregated into cellular clumps with trypsin and then transferred to a 1 cm culture dish with embryonic fibroblast feeder as previously described for isolation of mouse ES cells (Robertson, 1987). The cultures were fed every three or four days. After two to four weeks, colonies of adherent fibroblastic cells with loosely attached rapidly dividing round cells and characteristic ring-like cells were observed in some of the cultures [FIG. 1A]. These colonies were picked and expanded. The cells were maintained initially on embryonic fibroblast feeder plates and once a line was established, they were adapted to grow on gelatin-coated plates. For the older embryos, the egg cylinders were plated on a 24 well-plate with embryonic fibroblast feeder at one embryo per well. After a week or when the well was confluent, the well was trypsinized and the contents were transferred sequentially to a 48-well plate, 24-well plate, 6-well-plate and then a 10 cm plate. Monoclonal cell lines were established by either picking single colonies or plating single cells in methycellulose-based media. Chromosomes were counted as previously described (Robertson, 1987). Y chromosome FISH analysis was performed using mouse y-chromosome-specific probe from Cambio, Cambridge, UK.

Genomic DNA and total RNA analysis by PCR: Genomic DNA and total RNA were prepared using standard protocols and were quantified using, respectively, the RiboGreen RNA Quantification kit and the PicoGreen dsDNA Quantification kit (Molecular Probes, Eugene, Oregon). Primer set for Sry gene amplification were 5′-AGA GAT CAG CAA GCA GCT GG-3′ (SEQ ID NO:1) and 5′-TCT TGC CTG TAT GTG ATG GC-3′ (SEQ ID NO:2) and the expected amplified fragment size was 249 bp. PCR and RT-PCR was performed as previously described (Lim et al., 1998). The primer sets for each mRNA and its expected RT-PCR product were listed in FIG. 4A. All RT-PCR primers span at least one intron. In vitro differentiation: To differentiate RoSH2 cells in vitro, non-adherent cells from ˜80% confluent RoSH2 cell culture were collected and plated on a matrigel coated plate at 10⁵ cells per 6 cm tissue culture dish. To coat the plate with Matrigel, Matrigel (BD laboratories) was diluted 10× with ES media before spreading on a tissue culture dish and removing any excess Matrigel. The RoSH2-derived tubular structures were labelled with fluoresecent β-gal-specific substrate, Imagene Green™ according to the manufacturer's protocol (Molecular Probe, Eugene, Oreg.). Briefly, the substrate was added to the culture media at a final concentration of 33 μM and the culture was incubated overnight. The culture was fixed in formalin and counterstained with PI for nuclear staining.

Immunohistochemistry: Immunofluorescence and immunohistochemistry was performed using standard procedures. Cells and tissues were fixed in 4% paraformaldehyde. Tissues were embedded in paraffin and sectioned at 4 μm thickness. The primary antibodies used were: goat anti-mouse P-Selectin, goat anti-mouse CD34, rabbit anti-mouse TIE2, and rabbit anti-mouse Thy-1 (Santa Cruz Biotechnology, Inc, Santa Cruz, Calif.), rat anti-mouse CD31, rat anti-mouse Ly-6A/E (Sca-1) and rat anti-mouse Flk-1 (BD Pharmingen, San Diego, Calif.). The primary antibodies were detected using biotinylated secondary antibodies and strepavidin-conjugated horseradish peroxidase and DAB (Sigma, St Louis, Mo.). The sections were counterstained with Mayer's hematoxylin. For antibodies that did not react with paraffin-embedded sections, whole mount in-situ immunofluorescence was performed. Briefly, the tubular mesh was fixed in 4% paraformaldehyde and incubated in sequential order: the first primary antibody, a biotinylated secondary antibody and then avidin-FITC. The tissues were then counterstained with propidium iodide. The tubular mesh was analyzed by confocal microscopy.

Vascularization of ES cell-derived teratomas: For determining vascularization by RoSH2 in ES cell-derived teratoma, a cellular mix of 1×10⁴ RoSH2 cells and 1×10⁶ CS-ES cells (a gift from CS Lin) was injected subcutaneously into Rag1-/-mice. After 4-6 weeks, the mice were sacrificed and the tumors removed and cryosectioned at 12 μm for immunohistochemistry staining with rabbit anti-β-gal antibodies (ICN, Auroa, Ohio).

Incorporation of RoSH2 cells into liver vasculature during liver injury: To demonstrate incorporation of RoSH2 cells into the vasculature during liver injury, mice were anesthetized with 0.1 ml of a cocktail consisting of 1 part Hypnorm, 1 part Midazolom and 2 parts distilled water per 10 gm of body weight. 1×10⁶ RoSH2 cells were injected intrasplenically in 20 μl volume of saline. In sham-transplanted mice, saline was injected in the place of cells. The mice were then injected i.p. with 0.2 μg per g body weight of hamster anti-fas antibody (BD Pharmingen, San Diego, Calif.). After 5 days, the mice were perfused with saline followed by 0.5 % (v/v) glutaraldehyde. The livers were removed and soaked in 30% (w/v) sucrose in PBS and 0.5% glutaraldehyde overnight at 4° C. Whole livers or 12 μm thick cryosections were immersed in X-gal staining solution and incubated at 37° C. for 2-3 hours as previously described (Mercer et al., 1991). After staining, the livers or sections were washed with PBS. Tissue sections were mounted on slides and counterstained with hematoxylin and eosin (H&E).

RoSH2-derived lymphoid cells in Rag1-/-mice: 1×10⁶ RoSH2 cells were injected i.p. into six weeks old Rag1-/-mice. After six months, the mice were sacrificed, perfused with saline followed by 4% paraformaldehyde and the spleens and lungs were removed. The tissues were embedded in paraffin and sectioned at 4 μm thickness and analyzed for the presence of CD3+ cells by immunohistochemistry. The sections were counterstained with H&E.

RoSH2-derived myeloid and erythroid cells in vivo: Six weeks old C57BL6/J mice were treated with two doses of either 150 or 300 mg/Kg body of 5-fluorouracil at 24 hours apart. Twenty-four hours later, the mice were injected i.p. with 1×10⁶ RoSH2 cells. After ten weeks, the surviving mice were sacrificed and the spleens removed for colony assay. The assay was performed using a methycellulose-based assay, MethoCult™ that supports the growth of CFU-E, BFU-E, CFU-GM, CFU-G, CFU-M and CFU-GEMM (StemCell Technologies, Vancouver, Canada). The assay was performed according to manufacturer's protocol. Briefly, after red cell lysis by ammonium chloride, 1000 spleen cells were plated in 3 ml of the methycellulose-based assay medium. Two weeks later, the individual colonies are picked, washed with PBS, fixed with formalin, stained with Imagene Green™ (Molecular Probe, Eugene, Or) and counterstained with propidium iodide. The cells were analyzed by confocal microscopy.

Derivation of ROSH Cell Lines

As hemangioblasts are progenitor cells that give rise to both endothelial and hematopoietic cells, applicant postulated that these cells are likely to be present even before hematopoiesis and vasculogenesis are initiated in the yolk sac of E7 mouse embryos. To isolate putative hemangioblasts from embryos, 3.5 dpc blastocysts and 5.5 dpc delayed blastocysts were harvested. The delayed blastocysts were harvested from pregnant transgenic B6.129S7-GtRosa26 mice as described herein, and cultured on normal gelatinized tissue culture plate in ES media. 54 blastocysts were harvested, 20 hatched and showed some cell proliferation. After two to three weeks, the outgrowths were disaggregated with trypsin and plated on 24-well plates with mitomycin C-treated mouse embryonic fibroblast feeder plates. The cultures were maintained with changes of fresh media every two to four days. After two to four weeks, colonies of fibroblastic cells were observed in some of the cultures (FIG. 1A). From the first batch of 54 blastocysts, there were established two cell lines, RoSH1 and RoSH2 and they were adapted to grow on gelatinized plates. Lines that were derived from delayed 5.5 dpc blastocysts usually arose from one colony per blastocyst.

Although d3.5 and delayed d5.5 blastocysts are routinely used for the isolation of ES cells, applicant was able to isolate RoSH cell lines from only delayed blastocysts and not d3.5 blastocysts. To determine the upper limit of embryonic development from which isolation of these putative hemangioblasts was technically convenient, E6.0 to E7.5 embryos were isolated and the egg cylinders were dissected out. The egg cylinders were cultured on gelatinized, embryonic fibroblast feeder plates. It was observed that if the embryo proper was dissected for culture, growth of the embryo was poor but this can be remedied by placing the extraembryonic tissues besides the embryo. Once the embryo attached to the tissue culture plate and began to proliferate, the extraembryonic tissues were removed. By this means, hemangioblast lines have been isolated with relative ease from embryos as old as E7.0. Cultures of older embryos tend to produce complex mixtures of cell types. The frequency of deriving RoSH cell lines from delayed blastocysts was about 1 in 30 and that from E6.0 and older embryos was higher at about 1 in 10. Using delayed blastocysts to derive ROSH cell lines has the advantage of simplicity in establishing monoclonal lines. In all instances when ROSH lines were derived from delayed blastocysts, these lines arose from a single colony. Cultures of older embryos were complex with many different cell types and derivation of RoSH lines required extensive subcloning.

Once the line is established, the cells can be adapted to grow in ES media on gelatinized, feeder-free culture plates. To ensure clonality of the lines, single cells from each line were plated in methylcellulose-based media. After a week, colonies that were visible to the naked eye were picked and expanded. The cells have a fibroblastic morphology at sub-confluency with 1-5% of the cells assuming a vWF reactive ring-like structure (FIGS. 1A, 1D). At high confluency on gelatin-coated plates, the RoSH2 cells formed a meshwork of cord-like structures (FIG. 1B). Since RoSH2 cell line was derived from a B6.129S7-GtRosa26 embryo, it expressed β-galactosidase (β-gal) in the cytoplasm. This was verified by incubating the cells with Imagene Green™, a green fluorescent substrate for β-gal (FIG. 1C). One of the lines, RoSH2 has been maintained in continuous culture for more than 100 generations with a stable population doubling time of 12 hours and a stable morphology. At passages <10, the cell line has an euploid chromosomal complement with a strong modal number of 40. After more than 100 passages in continuous culture, there was a downward drift in modal number to about 35 (FIG. 1E). RoSH2 cells have a XY karyotype as verified by y-chromosome FISH and the presence of SRY gene by PCR (FIGS. 1F, 1G). All the lines tested by the applicant had a XY karyotype. At least 19 lines have been isolated with each line originating from a single embryo. Monoclonal sublines have been established for at least three of the lines and most of the experiments described were done with one line, RoSH2.

The mouse embryonic cell line designated RoSH2 was deposited at the American Type Culture Collection Patent Depositary (10801 University Blvd., Manassas, Va. 20110-2209, U.S.A.), and was assigned Patent Deposit Designation # PTA-4300 on May 29, 2002. This cell line is illustrative only of the mammalian hemangioblast cell lines which can be obtained by the method of the invention.

Differentiation of RoSH2 Cells

RoSH2 cells can be induced to differentiate to form a mesh of tubular structures by plating at a density of 1×10⁵ cells on 6-cm tissue plates that were thinly coated with matrigel. The formation of tubular structures varied with each batch of matrigel. On some occasions, lines of single cells were first observed to criss-cross across the tissue culture plates before they organized into tubular structures possibly through cell division (FIG. 2A). Usually, the cells proliferated to a high cell density before a network of tubular structures became visible. Sections of the tubular structures showed characteristic endothelial cells with flattened morphology lining a lumen (FIG. 2B). These tubular structures were enveloped by a layer of acellular matrix. The constituents of this matrix have not been identified. Clusters of round cells were loosely attached to the outer periphery of the tubular structures (FIG. 2B). There was also extensive branching and sprouting from the main tubular structures indicating that angiogenesis was also an integral component of ROSH differentiation (FIG. 2B). To determine the diameter and patency of the tubular structures, the mesh was incubated with Imagene Green™, a fluorescent substrate for β-gal, counterstained with propidium iodide and analyzed using confocal microscopy (FIG. 2C). By optical sectioning through the mesh, the lumens were verified to be patent with an average diameter of 30-50 μM and ranging up to 100-150 μM. Like endothelial cells, these cells endocytosed acetylated LDL (FIG. 2C). Electron microscopic examination of these tubular structures also suggested that these tubules were endothelial tubules (FIG. 2D). Neighbouring cells lining the lumen were in tight apposition and resting on an amorphous matrix. The plasma membrane was polarized with filamentous structures on the luminal surface. Intracellular microvesicles underlay the plasma membrane, suggesting endocytosis. Electron dense organelles were observed that were reminiscent of nascent Weibel-Palade bodies as previously described in endothelial cells generated from ES cell-derived hemangioblast (Choi et al., 1998).

The mesh of tubular structures usually covered the entire base of the plates. About one or two weeks after plating, the mesh began detaching from the center of the plate but the mesh remained strongly anchored at the perimeter. By rimming the plate with a 21G needle, the mesh with a relatively high tensile strength can be peeled off the plate in a single sheet with a pair of forceps, leaving a monolayer of undifferentiated RoSH2 cells. In one or two days, the remaining RoSH2 cells would sometimes form another mesh of tubular structures that was generally less extensive but with larger lumens.

Verification of RoSH2-Derived Endothelial and Hematopoietic Lineages by Immunohistochemistry

To verify that ROSH cells can differentiate into both endothelial and hematopoietic lineages, endothelial and hematopoietic markers were tested on the undifferentiated RoSH2 cells by FACS analysis and their tubular derivative cells by immunohistochemistry staining. These markers included CD34, PECAM-1 (or CD31), Flk-1, TIE2, Sca-1, Thy-1, CD45, P-selectin, and smooth muscle actin (FIG. 3). With the exception of TIE2, none of these markers were detected on the undifferentiated RoSH2 cells (FIG. 3A). TIE2 expression was detected on ˜5% of the undifferentiated cells and was relatively low. Immunohistochemistry staining of differentiated RoSH2 cells demonstrated that the endothelial and hematopoietic markers, CD34, PECAM-1 (or CD31), Flk-1, Tie-2, Sca-1, Thy-1, and P-selectin, were restricted to the surface membrane of cells forming the tubular structures (FIGS. 3B, 3C). CD45 expression was limited to round cells on the periphery of the tubular structures (FIG. 3B). These CD45+ve cells were rare but their presence was always unambiguous with strong membrane-localized CD45-specific staining. Some of the cells on the outer periphery of the tubular structures expressed smooth muscle actin in the cytoplasm, suggesting that these cells may be pericytes (FIG. 3C).

Gene Expression Profile of RoSH2 and Derivative Cells

To identify some of the genes that are involved in the maintenance of the ROSH stem-cell phenotype and in the differentiation of ROSH cells, total RNA was prepared from undifferentiated ROSH cells, ROSH cells grown on matrigel for 24 hours and ROSH cell-derived tubular structures and analyzed by RT-PCR (FIG. 4). Triose phosphate isomerase, a housekeeping gene, was used as control for RNA loading. Rex-1, a zinc-finger transcription factor regulated by Oct3/4 (Rogers et al., 1991), was expressed in RoSH2 cells. Its expression is associated with pluripotent stem cells such as ES cells and germ cells (Rogers et al., 1991) and is downregulated during differentiation of ES cell. There was a downregulation of Rex-1 in RoSH2 cells 24 hours after plating on matrigel (FIG. 4B). However, its expression continued to be detected in RNA prepared from RoSH2-derived tubular mesh. In situ hybridization demonstrated that expression of Rex-1 in the tubular structures was confined to round stem-like cells on the outer periphery of the tubes. Consistent with the potential of RoSH2 to differentiate into endothelial and hematopoietic cells, Brachyury mRNA was present in all three RNA preparations (FIG. 4C). Brachyury is a mesodermal lineage-specific gene and its expression indicated mesodermal commitment (Herrmann and Kispert, 1994; Smith, 1997; Technau, 2001).

As determined by RT-PCR, important transcription factors in the regulation of vasculogenesis and hematopoiesis such as SCL, PU.1, c-vav and GATA-1, angiogenic or hematopoietic growth factors and their receptors such as VEGF, VEGF receptor, Flk-1 and erythropoietin receptor were also expressed in the undifferentiated and differentiated RoSH2 cells (Keller, 2001; Orkin, 2001). Angiopoietin-1 mRNA was not detectable in the undifferentiated cells but was upregulated at least 10-fold during differentiation. Like angiopoietin, erythropoietin expression was also upregulated in the differentiated tubular structures. The presence of erythropoietin and erythropoietin receptor during differentiation of RoSH2 cells strongly suggests that RoSH2 cells could differentiate into erythrocytes. Expression of embryonic β-globin and adult β^{maj}-globin genes during differentiation of RoSH2 cells was therefore analyzed. Adult β^{maj}-globin gene but not embryonic β-globin was expressed (FIG. 4C), suggesting that definitive hematopoiesis predominated during RoSH2 differentiation. The gene expression profile of RoSH2 before and after differentiation was consistent with that in the differentiation of endothelial and hematopoietic cells. Since the SMA antibody that was used to detect smooth muscle actin in some of the differentiated RoSH2 cells by immunohistochemical staining occasionally cross-reacted with other actins (data not shown), the presence of SMA was further confirmed by RT-PCR (FIG. 4B). In all RT-PCR assays, the RT-PCR products were purified and sequenced for verification.

Transplantation of RoSH2 Cells in Mice to Generate Vascular Tissues

To determine if RoSH2 cells can differentiate into vascular tissues in vivo, RoSH2 cells were either subcutaneously co-injected with ES cells into B6.Rag1-/-mice or intrasplenically injected into C57BL6/J mice that were subsequently treated with anti-fas antibody to induce liver injury.

By co-injecting Rosa 2 cells with ES cells, applicant hypothesized that during in vivo differentiation of ES cells into tetratoma, the embryonic-like microenvironment of the developing teratoma will enable the RoSH2 cells to differentiate into its entire repertoire of potential cell derivatives. Co-injection of ES cells with RoSH2 cells subcutaneously into Rag1-/- mice produced highly vascularized, hemangioma-like tumors of about 1 cm diameter in two weeks (FIG. 5A). Co-injections of ES cells with RoSH2 resulted in the formation of tumors in 4 out of 6 mice within 3-4 weeks while tumors from injections of ES cells alone will reach similar sizes only after 4-6 weeks. Subcutaneous or intramuscular injections of RoSH2 cells into isogenic C57BL6/J or B6.Rag1-/-mice did not cause any tumor formation during a six months observation period. Tumors formed by co-injections of RoSH2 and ES cells were essentially highly vascularized tumors sometimes with cavernous hemangiomas (FIG. 5A). RoSH2-derived endothelial cells as evident by β-gal immunohistochemistry staining were detected in some of the vascular structures filled with blood (FIG. 5A). Although tumors formed from ES cells alone were also vascularized, they were often hard solid tumors.

The possibility that RoSH2 cells can also participate in vascular remodelling during tissue injury in adults was also tested. Mice were injected intrasplenically with RoSH2 cells and then treated with anti-fas antibody to induce liver damage. Ten days later, livers from these mice were removed and stained for the presence of β-gal to determine incorporation of RoSH2 cells into the tissues. Whole mount staining showed extensive incorporation of the cells in the regenerating liver and further microscopic analysis demonstrated that RoSH2 cells were incorporated into the endothelium of the liver vasculature (FIG. 5B).

RoSH2 Cells Differentiate into Myeloid and Lymphoid Tissues in vivo

In vitro differentiation of RoSH2 cells suggests that these cells can generate hematopoietic cells. To verify this in vivo, RoSH2 cells were injected intraperitoneally into six weeks old Rag1-/- mice. Rag1-/- mice are “non-leaky” severe combined immune deficiency mice that do not have any mature CD3+ T-cell (Mombaerts et al., 1992). At six months, spleens were removed and stained for the presence of CD3+ cells (FIG. 6A). Distinctly membrane-localized CD3+ lymphoid cells were present.

To determine if the RoSH2 cells can form myeloid cells, 30 C57BL6/J mice were treated with 5-fluorouracil as described herein, and transplanted with RoSH2 cells. 28 of the mice died within the first two weeks. At ten weeks, the mice were sacrificed and their spleens were harvested and assayed for the presence of myeloid progenitor cells by colony forming unit assay. Eleven colonies were picked, the cells were stained with Imagene Green™ to assay for the presence of β-gal and the nuclei were counterstained with propidium iodide (FIG. 6B). Six colonies were positive for β-gal indicating that these myeloid cells must be derived from the transplanted RoSH2 cells. Two of the eleven colonies were mixed colonies with both megakaryocytes and erythrocytes, four were erythrocyte colonies, three were megakaryocyte colonies and the remaining two appeared to be colonies of monocyte.

### EXAMPLE 2

Preparation of Hemangioblast Cell Lines from Embryonic Stem Cells

2×10⁴ single ES cells in 100 μl ES media ES cells were cultured in 3.9 ml methycellulose media (MethoCult M3134, StemCell Technologies, Inc, Vancouver, Canada), 4.2 ml IMDM (Life Technologies, Rockville, Md.), 1.5 ml Serum, 100 μl monothioglycerol stock solution (37.8 μl in 10 ml PBS) (Sigma, St Louis, Mo.) 100 μl 100X L-glutamine/Penicillin/Streptomycin stock solution (Life Technologies, Rockville, Md.). Six days later, colonies of cells or EBs were clearly visible to the naked eyes. The EBs were then dissociated into cell suspensions by incubating the EBs in 0.15% (w/v) collagenase/PBS supplemented with 20% (v/v) FCS at 37° C. for 30 minutes and then disrupting the cell clumps by passing the solution through a syringe with a 20 -gauge needle 3 times. After another 30 minutes of incubation, the disruption was repeated with a 25-gauge needle. These cells were then plated on mitomycin C-treated embryonic fibroblast at a density of 1-5×20⁵ cells per 10 cm gelatin-coated plate. After about a week, the cells proliferated and differentiated into a complex mixture of cell types. Colonies of rapidly dividing cells resembling embryo-derived hemangioblast cells were picked, pooled, diluted to one cell per 100 μl and plated at 100 μl/well on a 96-well feeder plate. From 10×96-well plates, applicant was able to establish at least 5 lines. The cells were maintained initially on embryonic fibroblast feeder plates and once a line was established, it was adapted to grow on gelatin-coated plates.

Another convenient way of isolating hemangioblast cell lines from EBs is to pick individual EBs and place each EB per well in a gelatinized 96-well feeder plate. Each EB then adhered to the culture dish. After several days of proliferation, putative hemangioblast colonies were present in at least 50% of the wells. The colonies were then picked and expanded. In most instances, the EBs would proliferate into a complex mixture of cells. By serially expanding these cells on feeder cells, most of the cells reached senescence and died, leaving one or two cell types which can then be picked and expanded.

### EXAMPLE 3

Preparation of Hemangioblast Cell Lines from Bone Marrow

Adult bone marrow (BM) was prepared from mice, pigs and humans. For mice, BM was flushed from the femurs of B6.129S7-GtRosa26 with saline using a needle and syringe. In pigs, BM was aspirated from the femur of pigs. Human BM was harvested by scraping from the split sternum of patients undergoing CABG surgery at NUH. The common denominator in all these procedures is the preservation of some BM tissue integrity in tissue clumps of 0.1 to 1 mm³ in volume. Each piece of tissue was cultured individually on 48-well mitomycin C-treated mouse embryonic fibroblast feeder plates in ES cell media. Most of the BM pieces attached to the plates within 24 hours. The cultures were maintained with changes of fresh media every two to four days. Over a period of one week, cells appeared to migrate out of the BM pieces. During the first week, the culture was a complex mix of cell types with much cell proliferation and cell death occurring simultaneously. After one to two weeks, distinct colonies of adherent fibroblastic cells with loosely attached, rapidly dividing round cells and characteristic ring-like cell were observed in some of the cultures (FIG. 8).

They are highly typical of the hemangioblast colonies that were previously isolated from mouse embryos and ES cells. These colonies appeared at a frequency of one per 15 pieces. Once a cell culture was established, i.e. maintained in continuous culture for 20 generations, the cells can be adapted to grow in ES media on gelatinized, feeder-free culture plates. To clone the cells, single cells were plated in methylcellulose-based media.

The cells derived from mouse BM were named Ro(BM)SH, those from human BM were named HuSH and those from pig BM were named PoSH. Each name is followed by a number to indicate their derivation from an independent source of bone marrow.

The characteristic ring-like cells from both mouse and human BM were vWF reactive. Like embryo-derived hemangioblast cell line, RoSH2 cells, each of Ro(BM)SH, HuSH and PoSH cells formed a meshwork of cord-like structures at high confluency on gelatin-coated plates. They have a population doubling time of about 15 hours. Like undifferentiated RoSH2 cells, Ro(BM)SH is not immunoreactive with antibodies specific for markers of pluripotent cells including CD34, PECAM-1 (or CD31), Flk-1, TIE2, Sca-1, Thy-1, CD45 and P-selectin by FACS analysis. The proportion of cells that were positive for these markers corresponded with the approximate proportion of ring-like cells in the cell population, suggesting that these markers were detectable only on differentiated cells (FIG. 9).

This is consistent with the immunohistochemical analysis of differentiated ROSH cells where it was demonstrated that after in vitro differentiation of ROSH cells to form vascular structures, these vascular structures expressed detectable levels of these markers.

### EXAMPLE 4

In vitro Differentiation

Like RoSH2 cells, Ro(BM)SH and HuSH cells can be induced to differentiate to form a mesh of tubular structures by plating at a density of 1×10⁶ cells on 6-cm tissue plates that were thinly coated with matrigel.

### EXAMPLE 5

Gene Expression Profile of Ro(BM) and HuSH

Total RNA from undifferentiated and differentiated Ro(BM)SH cells were analyzed by RT-PCR. Triose phosphate isomerase, a housekeeping gene was used as control for RNA loading. Markers that are associated with pluripotent stem cells such as ES cells and germ cells e.g. Rex-1, a zinc-finger transcription factor regulated by Oct3/4 regulated and Oct3/4 transcription factor, were expressed in Ro(BM)SH. Gene expression in Ro(BM)SH cells mirrored that of embryo-derived RoSH cells. When compared mouse ES cells, expression of Rex-1 and Oct3/4 was much reduced. Like ROSH cells, Brachyury, a mesodermal lineage-specific gene was also upregulated in Ro(BM)SH. In addition, important transcription factors in the regulation of vasculogenesis and hematopoiesis such as SCL, PU.1, c-vav and GATA-1, angiogenic or hematopoietic growth factors and their receptors such as VEGF, VEGF receptor, Flk-1 and erythropoietin receptor were also expressed. Together with cell morphology, cell growth pattern and surface antigen profile, this gene expression profile suggests that BM-derived Ro(BM)SH cells are similar to embryo-derived ROSH cells, i.e. they are mesodermal stem cells with endothelial and hematopoietic potentials (FIG. 10).

Although the foregoing invention has been described in detail for the purposes of clarity of understanding, it will be obvious that certain modifications can be practised within the scope of the appended claims. All publications and patent documents cited herein are hereby incorporated by reference in their entirety for all purposes to the same extent as if each were so individually denoted.

### REFERENCES

Bahary, N., and Zon, L. I. (2001). Development. Endothelium—chicken soup for the endoderm. Science 294, 530-531.

Bustelo, X. R., et al. (1993). Developmental expression of the vav protooncogene. Cell Growth Differ 4, 297-308.

Carmeliet, P., et al. (1996). Abnormal blood vessel development and lethality in embryos lacking a single VEGF allele. Nature 380, 435-439.

Choi, K., et al. (1998). A common precursor for hematopoietic and endothelial cells. Development 125, 725-732.

Davis, S., et al. (1996). Isolation of angiopoietin-1, a ligand for the TIE2 receptor, by secretion-trap expression cloning. Cell 87, 1161-1169.

Doetschman, T. C., et al. (1985). The in vitro development of blastocyst-derived embryonic stem cell lines: formation of visceral yolk sac, blood islands and myocardium. J Embryol Exp Morphol. 1985; 87:27-45.

Dzierzak, E. (1999). Embryonic beginnings of definitive hematopoietic stem cells. Ann N Y Acad Sci 872, 256-262; discussion 262-254.

Ferrara, N., et al. (1996). Heterozygous embryonic lethality induced by targeted inactivation of the VEGF gene. Nature 380, 439-442.

Fischer, K. D., et al. (1995). Defective T-cell receptor signalling and positive selection of Vav-deficient CD4+ CD8+ thymocytes. Nature 374, 474-477.

Godin, I., et al. (1995). Emergence of multipotent hemopoietic cells in the yolk sac and paraaortic splanchnopleura in mouse embryos, beginning at 8.5 days postcoitus. Proc Natl Acad Sci U S A 92, 773-777.

Hamaguchi, I., et al. (1999). In vitro hematopoietic and endothelial cell development from cells expressing TEK receptor in murine aorta-gonad-mesonephros region. Blood 93, 1549-1556.

Herrmann, B. G., and Kispert, A. (1994). The T genes in embryogenesis. Trends Genet 10, 280-286.

Hromas, R., et al. (1993). Hematopoietic lineage- and stage-restricted expression of the ETS oncogene family member PU.1. Blood 82, 2998-3004.

Jiang, Y., et al. (2002). Pluripotency of mesenchymal stem cells derived from adult marrow. Nature 418, 41-49.

Katzav, S., et al. (1989). vav, a novel human oncogene derived from a locus ubiquitously expressed in hematopoietic cells. Embo J 8, 2283-2290.

Keller, G. (2001). The Hemangioblast. In Stem Cell Biology, D. R. Marshak, R. L. Gardener, and D. Gottlieb, eds. (Cold Spring Harbor, Cold Spring Harbor Laboratory Press), pp. 329-348.

Keller, G., et al. (1993). Hematopoietic commitment during embryonic stem cell differentiation in culture. Mol Cell Biol 13, 473-486.

Keller, G., et al. (1999). Development of the hematopoietic system in the mouse. Exp Hematol 27, 777-787.

Kennedy, M., et al. (1997). A common precursor for primitive erythropoiesis and definitive haematopoiesis. Nature 386, 488-493.

Lammert, E., et al. (2001). Induction of pancreatic differentiation by signals from blood vessels. Science 294, 564-567.

Levenberg S., et al. (2002) Endothelial cells derived from human embryonic stem cells. Proc Natl Acad Sci U S A. 99, 4391-4396.

Lim, S. K., et al. (1998). Increased susceptibility in Hp knockout mice during acute hemolysis. Blood 92, 1870-1877.

Lin, C. S., et al. (1996). Differential effects of an erythropoietin receptor gene disruption on primitive and definitive erythropoiesis. Genes Dev 10, 154-164.

Matsumoto, K., et al. (2001). Liver organogenesis promoted by endothelial cells prior to vascular function. Science 294, 559-563.

McKercher, S. R., et al. (1996). Targeted disruption of the PU.1 gene results in multiple hematopoietic abnormalities. Embo J 15, 5647-5658.

Mercer, E. H., et al. (1991). The dopamine beta-hydroxylase gene promoter directs expression of E. coli lacZ to sympathetic and other neurons in adult transgenic mice. Neuron 7, 703-716.

Mombaerts, P., et al. (1992). RAG-1 deficient mice have no mature B and T lymphocytes. Cell 68, 869-877.

Moore, M. S. A., and Metcalf, D. (1970). Ontogeny of the hematopoietic system: Yolk sac origin of in vivo and in vitro colony forming cells in the mouse embryo. Br J Hematology 18, 279-296.

Muller, A. M., et al. (1994). Development of hematopoietic stem cell activity in the mouse embryo. Immunity 1, 291-301.

Murray, P. D. F. (1932). The development in vitro of the blood of the early chick embryo. Proc Roy Soc London 11, 497-521.

Nerlov, C., and Graf, T. (1998). PU.1 induces myeloid lineage commitment in multipotent hematopoietic progenitors. Genes Dev 12, 2403-2412.

Nishikawa, S. I., et al. (1998). Progressive lineage analysis by cell sorting and culture identifies FLK1+VE-cadherin+ cells at a diverging point of endothelial and hemopoietic lineages. Development 125, 1747-1757.

Orkin, S. H. (2001). Hematopoietic Stem Cells: Molecular Diversification and Developmental Interrelationships. In Stem Cell Biology, D. R. Marshak, R. L. Gardener, and D. Gottlieb, eds. (Cold Spring Harbor, Cold Spring Harbor Laboratory Press).

Pevny, L., et al. (1991). Erythroid differentiation in chimaeric mice blocked by a targeted mutation in the gene for transcription factor GATA-1. Nature 349, 257-260.

Robb, L., et al. (1996). The scl gene product is required for the generation of all hematopoietic lineages in the adult mouse. Embo J 15, 4123-4129.

Robertson, E. J. (1987). Embryo-derived stem cell lines. In Teratocarcinomas and embryonic stem cells: a practical approach., E. J. Robertson, ed. (Oxford, IRL Press Limited), pp. 71-112.

Rogers, M. B., et al. (1991). Specific expression of a retinoic acid-regulated, zinc-finger gene, Rex-1, in preimplantation embryos, trophoblast and spermatocytes. Development 113, 815-824.

Sabin, E. R. (1920). Studies on the origin of blood vessels and of red corpuscles as seen in the living blastoderm of the chick during the second day of incubation. Contributions to Embryology 9, 213-262.

Scott, E. W., et al. (1994). Requirement of transcription factor PU.1 in the development of multiple hematopoietic lineages. Science 265, 1573-1577.

Shalaby, F., et al. (1995). Failure of blood-island formation and vasculogenesis in Flk-1-deficient mice. Nature 376, 62-66.

Simon, M. C., et al. (1992). Rescue of erythroid development in gene targeted GATA-1- mouse embryonic stem cells. Nat Genet 1, 92-98.

Smith, J. (1997). Brachyury and the T-box genes. Curr Opin Genet Dev 7, 474-480.

Suri, C., et al. (1998). Increased vascularization in mice overexpressing angiopoietin-1. Science 282, 468-471.

Takakura, N., et al. (1998). Critical role of the TIE2 endothelial cell receptor in the development of definitive hematopoiesis. Immunity 9, 677-686.

Tarakhovsky, A., et al. (1995). Defective antigen receptor-mediated proliferation of B and T cells in the absence of Vav. Nature 374, 467-470.

Technau, U. (2001). Brachyury, the blastopore and the evolution of the mesoderm. Bioessays 23, 78-794.

Wiles, M. V. (1993) Embryonic stem cell differentiation in vitro. Methods in Enzymology. 225, 900-918.

Wu, H., et al. (1995). Generation of committed erythroid BFU-E and CFU-E progenitors does not require erythropoietin or the erythropoietin receptor. Cell 83, 59-67.

1. A purified preparation of mammalian hemangioblast cells which (i) is capable of proliferation in an in vitro culture for more than 40 generations, (ii) does not induce tumor formation in an immunodeficent Rag1 deficient mouse, (iii) maintains the potential to differentiate to hematopoietic and endothelial cells throughout the duration of said culture, and (iv) are inhibited from differentiation when cultured on a gelatinized, feeder-free layer.

2. The preparation of claim 1, wherein the cells are not immunoreactive with CD34, PECAM-1 (or CD31), Flk-1, Tie-2, Sca-1, Thy-1 and P-selectin markers.

3. The preparation of claim 1 wherein the cells are human.

4. The preparation of claim 2 wherein the cells are human.

5. The preparation of claim 1 wherein the mammalian hemangioblast cells are mouse embryonic cell line deposited under ATCC PTA-4300.

6. A method of preparing a mammalian hemangioblast cell line, comprising the steps of: (i) culturing on a feeder layer a cell source selected from the group consisting of a delayed mammalian blastocyst, an early post-implantation embryo together with its extra-embryonic tissues, an embryonic stem cell-derived embryoid body, and bone marrow tissue, (ii) selecting colonies of adherent fibroblastic cells with loosely attached rapidly dividing round cells having ring-like cells at their edges, and (iii) testing cells in the selected colonies for ability to differentiate into both endothelial and hematopoietic cells.

7. The method as claimed in claim 6, wherein the cell source is bone marrow tissue, and further comprising the step of harvesting bone marrow tissue which retains integrity in tissue clumps prior to the step of culturing.

8. The method as claimed in claim 6, wherein the cell source is human.

9. The method as claimed in claim 7, wherein the cell source is human.

10. The method as claimed in claim 6, further comprising maintaining the selected cells on a gelatinized feeder-free layer to inhibit differentiation.

11. The method as claimed in claim 7, further comprising maintaining the selected cells on a gelatinized feeder-free layer to inhibit differentiation.

12. A cell line developed by the method of claim 6.

13. A method for inducing formation of new blood vessels in an ischemic tissue in a patient in need thereof, comprising administering to said patient an effective amount of the purified preparation of mammalian hemangioblast cells according to claim 3 to induce new blood vessel formation in said ischemic tissue.

14. A method of enhancing blood vessel formation in a patient in need thereof, comprising: (i) selecting the patient in need thereof; (ii) isolating human hemangioblast cells according to the method of claim 8; and (iii) administering the hemangioblast cells to the patient.

15. A method for treating an injured blood vessel in a patient in need thereof, comprising: (i) selecting the patient in need thereof; (ii) isolating human hemangioblast cells according to the method of claim 8; and (iii) administering the hemangioblast cells to the patient.

16. A method of delivering a therapeutic gene to a patient having a condition amenable to gene therapy comprising: (i) selecting the patient in need thereof; (ii) modifying the preparation of claim 3 so that the cells of the preparation carry a therapeutic gene; and (iii) administering the modified preparation to the patient.

17. A commercial package comprising the preparation of claim 3 wherein the preparation has been modified so that the cells of the preparation carry a therapeutic gene, and instructions for treating a patient having a condition amenable to treatment with gene therapy.


SEQUENCE LISTING (44 sequences)

SEQ ID NO: 1 - DNA, 20 bp, Mus musculus
agagatcagcaagcagctgg

SEQ ID NO: 2 - DNA, 20 bp, Mus musculus
tcttgcctgtatgtgatggc

SEQ ID NO: 3 - DNA, 20 bp, Mus musculus
ccgcctcctcttcctccctg

SEQ ID NO: 4 - DNA, 20 bp, Mus musculus
cgtcgtattcctgtttgctg

SEQ ID NO: 5 - DNA, 20 bp, Mus musculus
gaagccaaggacagagacac

SEQ ID NO: 6 - DNA, 20 bp, Mus musculus
gcaacaacggaggacattag

SEQ ID NO: 7 - DNA, 20 bp, Mus musculus
gtataacgtggaggtcaagc

SEQ ID NO: 8 - DNA, 20 bp, Mus musculus
ggaggactggagaaaatcag

SEQ ID NO: 9 - DNA, 20 bp, Mus musculus
atgtcactgctggtgctgga

SEQ ID NO: 10 - DNA, 20 bp, Mus musculus
tgctagccaattcctcccag

SEQ ID NO: 11 - DNA, 20 bp, Mus musculus
cctatgaccacccacatccg

SEQ ID NO: 12 - DNA, 22 bp, Mus musculus
gatgaggaccagaatgagagac

SEQ ID NO: 13 - DNA, 19 bp, Mus musculus
cgctctgtggttctgcgtg

SEQ ID NO: 14 - DNA, 22 bp, Mus musculus
catccggaacaaatctcttttc

SEQ ID NO: 15 - DNA, 20 bp, Mus musculus
actcgtcataccactaaggt

SEQ ID NO: 16 - DNA, 20 bp, Mus musculus
agtgtctgtaggcctcagct

SEQ ID NO: 17 - DNA, 20 bp, Mus musculus
actgaccctaaagcaagacg

SEQ ID NO: 18 - DNA, 20 bp, Mus musculus
cagccatcaaaaggacacac

SEQ ID NO: 19 - DNA, 20 bp, Mus musculus
attgcacacacgggattctg

SEQ ID NO: 20 - DNA, 20 bp, Mus musculus
catacagtacgacactgacg

SEQ ID NO: 21 - DNA, 21 bp, Mus musculus
ccctggcatgatcaaagactt

SEQ ID NO: 22 - DNA, 21 bp, Mus musculus
gatgggcagtgctcattgttt

SEQ ID NO: 23 - DNA, 21 bp, Mus musculus
tgaactttctgctctcttggg

SEQ ID NO: 24 - DNA, 21 bp, Mus musculus
gttctgtctttctttggtctg

SEQ ID NO: 25 - DNA, 19 bp, Mus musculus
gggaggaaaaagagaagag

SEQ ID NO: 26 - DNA, 21 bp, Mus musculus
acttgctgctgcaacggagac

SEQ ID NO: 27 - DNA, 19 bp, Mus musculus
gggaggaaaaagagaagag

SEQ ID NO: 28 - DNA, 20 bp, Mus musculus
tgaaatcagcaccgtgtaag

SEQ ID NO: 29 - DNA, 20 bp, Mus musculus
tccaggtgctatatattgcc

SEQ ID NO: 30 - DNA, 20 bp, Mus musculus
tgctgcctgtgagtcataac

SEQ ID NO: 31 - DNA, 20 bp, Mus musculus
gaagccaaggacagagacac

SEQ ID NO: 32 - DNA, 20 bp, Mus musculus
gcaacaacggaggacattag

SEQ ID NO: 33 - DNA, 21 bp, Mus musculus
gagacaggcagcaagaaaaag

SEQ ID NO: 34 - DNA, 21 bp, Mus musculus
aatgaggggatggggaggaag

SEQ ID NO: 35 - DNA, 22 bp, Mus musculus
ggacagcatctggtgggtggac

SEQ ID NO: 36 - DNA, 22 bp, Mus musculus
cctcgcctgtcttgccgtagtt

SEQ ID NO: 37 - DNA, 20 bp, Mus musculus
aaggaaagaaggaaggaaag

SEQ ID NO: 38 - DNA, 20 bp, Mus musculus
gtccccatggagtcaaagag

SEQ ID NO: 39 - DNA, 19 bp, Mus musculus
gtccccatggagtcaaaga

SEQ ID NO: 40 - DNA, 20 bp, Mus musculus
ctcaaggagacctttgctca

SEQ ID NO: 41 - DNA, 20 bp, Mus musculus
ctgacagatgctctcttggg

SEQ ID NO: 42 - DNA, 20 bp, Mus musculus
cacaaccccagaaacagaca

SEQ ID NO: 43 - DNA, 21 bp, Mus musculus
tgactgatgctgagaaggctg

SEQ ID NO: 44 - DNA, 21 bp, Mus musculus
caggcaagagcaggaaagggg